(19) United States
(12) Patent Application Publication
(10) Pub. No.: US 2006/0173041 A1
(43) Pub. Date: Aug. 3, 2006

(54) Sulphonylpiperidine derivatives containing an aryl or heteroaryl group for use as matrix metalloproteinase inhibitors

(76) Inventors: Jeremy Nicholas Burrows, Cheshire (GB); Howard Tucker, Cheshire (GB); David Waterson, Cheshire (GB); Maurice Raymond Verschoyle Cheshire, Cheshire (GB)

Correspondence Address: FISH & RICHARDSON P.C., P.O BOX 1022, MINNEAPOLIS, MN, 55440-1022, US

(21) Appl. No.: 10/520,861

(22) PCT Filed: Jul. 9, 2003
(86) PCT No.: PCT/GB03/02982

§ 371 Date: Jan. 11, 2005

(30) Foreign Application Priority Data
Jul. 13, 2002 (GB) 0216383.0

(51) Int. Cl. A61K 31/445; A61K 31/454; C07D 401/02

(52) U.S. Cl. 514/317; 546/216; 514/326; 546/207

(57) ABSTRACT

A compound of formula (1), wherein B is an ortho substituted monocyclic aryl or heteroaryl group or a bicyclic aryl or heteroaryl group; useful in the inhibition of one or more metalloproteinases, and in particular TACE.

The present invention relates to compounds useful in the inhibition of metalloproteinases and in particular to pharmaceutical compositions comprising them, as well as their use.

The compounds of this invention are inhibitors of one or more metalloproteinase enzymes and are particularly effective as inhibitors of TACE (TNFα Convering Enzyme). Metalloproteinases are a superfamily of proteinases (enzymes) whose numbers in recent years have increased dramatically. Based on structural and functional considerations these enzymes have been classified into families and subfamilies as described in N. M. Hooper (1994) FEBS Letters 354:1-6. Examples of metalloproteinases include the matrix metalloproteinases (MMP) such as the collagenases (MMP1, MMP8, MMP13), the gelatinases (MMP2, MMP9), the stromelysins (MMP3, MMP10, MMP11), matrilysin (MMP7), metalloelastase (MMP12), enamelysin (MMP19), the MT-MMPs (MMP14, MMP15, MMP16, MMP17); the reprolysin or adamalysin or MDC family which includes the secretases and sheddases such as TNF converting enzymes (ADAM10 and TACE); the astacin family which include enzymes such as procollagen processing proteinase (PCP); and other metalloproteinases such as aggrecanase, the endothelin converting enzyme family and the angiotensin converting enzyme family.

Metalloproteinases are believed to be important in a plethora of physiological disease processes that involve tissue remodelling such as embryonic development, bone formation and uterine remodelling during menstruation. This is based on the ability of the metalloproteinases to cleave a broad range of matrix substrates such as collagen, proteoglycan and fibronectin. Metalloproteinases are also believed to be important in the processing, or secretion, of biologically important cell mediators, such as tumour necrosis factor (TNF); and the post translational proteolysis processing, or shedding, of biologically important membrane proteins, such as the low affinity IgE receptor CD23 (for a more complete list see N. M. Hooper et al., (1997) Biochem J. 321:265-279).

Metalloproteinases have been associated with many disease conditions. Inhibition of the activity of one or more metalloproteinases may well be of benefit in these disease conditions, for example: various inflammatory and allergic diseases such as, inflammation of the joint (especially rheumatoid arthritis, osteoarthritis and gout), inflammation of the gastro-intestinal tract (especially inflammatory bowel disease, ulcerative colitis and gastritis), inflammation of the skin (especially psoriasis, eczema and dermatitis); in tumour metastasis or invasion; in disease associated with uncontrolled degradation of the extracellular matrix such as osteoarthritis; in bone resorptive disease (such as osteoporosis and Paget's disease)); in diseases associated with aberrant angiogenesis; the enhanced collagen remodelling associated with diabetes, periodontal disease (such as gingivitis), corneal ulceration, ulceration of the skin, post-operative conditions (such as colonic anastomosis) and dermal wound healing; demyelinating diseases of the central and peripheral nervous systems (such as multiple sclerosis); Alzheimer's disease; and extracellular matrix remodelling observed in cardiovascular diseases such as restenosis and atheroscelerosis.

A number of metalloproteinase inhibitors are known; different classes of compounds may have different degrees of potency and selectivity for inhibiting various metalloproteinases. We have discovered a class of compounds that are inhibitors of metalloproteinases and are of particular interest in inhibiting TACE. The compounds of this invention have beneficial potency and/or pharmacokinetic properties.

TACE (also known as ADAM17) which has been isolated and cloned [R. A. Black et al. (1997) Nature 385:729-733; M. L. Moss et al. (1997) Nature 385:733-7361 is a member of the admalysin family of metalloproteins. TACE has been shown to be responsible for the cleavage of pro-TNFα, a 26 kDa membrane bound protein to release 17 kDa biologically active soluble TNFα. [Schlondorff et al. (2000) Biochem. J. 347: 131-138]. TACE mRNA is found in most tissues, however TNFα is produced primarily by activated monocytes, macrophages and T lymphocytes. TNFα has been implicated in a wide range of pro-inflammatory biological processes including induction of adhesion molecules and chemokines to promote cell trafficking, induction of matrix destroying enzymes, activation of fibroblasts to produce prostaglandins and activation of the immune system [Aggarwal et al (1996) Eur. Cytokine Netw. 7: 93-124]. Clinical use of the anti-TNF biologicals has shown TNFα to play an important role in a range of inflammatory diseases including rheumatoid arthritis, Crohn's disease and psoriasis [Onrust et al (1998) Biodrugs 10: 397-422, Jarvis et al (1999) Drugs 57:945-964]. TACE activity has also been implicated in the shedding of other membrane bound proteins including TGFα, p75 & p55 TNF receptors, L-selectin and amyloid precursor protein [Black (2002) Int. J. Biochem. Cell Biol. 34: 1-5]. The biology of TACE inhibition has recently been reviewed and shows TACE to have a central role in TNFα production and selective TACE inhibitor to have equal, and possibly greater, efficacy in the collagen induced arthritis model of RA than strategies that directly neutralise TNFα [Newton et al (2001) Ann. Rheum. Dis. 60: iii25-iii32].

A TACE inhibitor might therefore be expected to show efficacy in all disease where TNFα has been implicated including, but not limited to, inflammatory diseases including rheumatoid arthritis and psoriasis, autoimmune diseases, allergic/atopic diseases, transplant rejection, graft versus host disease, cardiovascular disease, reperfusion injury and malignancy.

Compounds that inhibit matrix metalloproteinases are already known in the art. WO 00/12477 discloses hydroxamic acids and carboxylic acid derivatives that are inhibitors of matrix metalloproteinases; WO 00/12478 discloses arylpiperazines that are useful in the inhibition of matrix metalloproteinases and are of particular interest as regards the inhibition of MMP13 and MMP9; and WO 01/87870 discloses hydroxamic acid derivatives which are inhibitors of matrix metalloproteinases including ADAM or ADAM-TS enzymes.

Surprisingly we have discovered that a selection of compounds are very potent inhibitors of TACE (ADAM17) and are particularly noteworthy for their unexpected selectivity for TACE over the matrix metalloproteinases

Additionally further effective compounds are disclosed.

According to one aspect of the present invention there is provided a compound of formula (1): 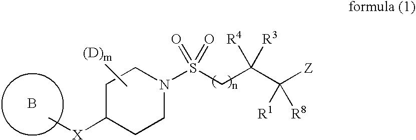
wherein Z is selected from —CONR¹⁵OH and —N(OH)CHO;
R¹⁵ is hydrogen or C₁₋₃alkyl;
wherein R¹ is hydrogen or a group selected from C₁₋₆alkyl, C₂₋₆alkenyl, C₂₋₆alkynyl, C₃₋₇cycloalkyl, C₅₋₇cycloalkenyl, aryl, heteroaryl and heterocyclyl where the group is optionally substituted by one or more substituents independently selected from halo, nitro, cyano, trifluoromethyl, trifluoromethoxy, C₁₋₄alkyl, C₂₋₄alkenyl, C₂₋₄alkynyl, C₃₋₆cycloakyl (optionally substituted by one or more R¹⁷), aryl (optionally substituted by one or more R¹⁷), heteroaryl (optionally substituted by one or more R¹⁷), heterocyclyl, C₁₋₄alkoxycarbonyl, —OR⁵, —SR², —SOR², —SO₂R², —COR², —CO₂R⁵, —CONR⁵R⁶, —NR¹⁶COR⁵, —SO₂NR⁵R⁶ and —NR¹⁶SO₂R²;
R¹⁶ is hydrogen or C₁₋₃alkyl;
R¹⁷ is selected from halo, C₁₋₆alkyl, C₃₋₆cycloalkyl and C₁₋₆alkoxy;
R² is group selected from C₁₋₆alkyl, C₃₋₆cycloalkyl, C₅₋₇cycloalkenyl, heterocycloalkyl, aryl, heteroaryl, arylC₁₋₄alkyl and heteroarylC₁₋₄alkyl where the group is optionally substituted by one or more halo;
R⁵ is hydrogen or a group selected from C₁₋₆alkyl, C₃₋₆cycloalkyl, C₅₋₇cycloalkenyl, heterocycloalkyl, aryl, heteroaryl, arylC₁₋₄alkyl and heteroarylC₁₋₄alkyl where the group is optionally substituted by one or more halo;
R⁶ is hydrogen, C₁₋₆alkyl or C₃₋₆cycloalkyl;
or R⁵ and R⁶ together with the nitrogen to which they are attached form a heterocyclic 4 to 7-membered ring;
wherein R⁸ is hydrogen or a group selected from C₁₋₆alkyl, C₃₋₇cycloalkyl, C₅₋₇cycloalkenyl and heterocyclyl where the group is optionally substituted by one or more substituents independently selected from halo, nitro, cyano, trifluoromethyl, trifluoromethoxy and C₁₋₄alkyl;
or R¹ and R⁸ together form a carbocyclic or saturated heterocyclic 3- to 6-membered ring; wherein R³ and R⁴ are independently hydrogen, C₁₋₆alkyl, C₃₋₆cycloalkyl, C₅₋₇cycloalkenyl, heterocyclyl, aryl or heteroaryl;
wherein n is 0 or 1;
wherein m is 0 or 1;
wherein D is hydrogen, C₁₋₄alkyl, C₃₋₆cycloalkyl or fluoro;
wherein X is O, S, SO or SO₂;
wherein B is monocyclic aryl or heteroaryl where each is substituted in an ortho position and is optionally further substituted by one or more groups independently selected from nitro, trifluoromethyl, trifluoromethoxy, halo, C₁₋₄alkyl (optionally substituted by R¹³), C₂₋₄alkenyl (optionally substituted by R¹³), C₂₋₄alkynyl (optionally substituted by R¹³), C₃₋₆cycloalkyl (optionally substituted by R¹³), C₃₋₆cycloalkenyl (optionally substituted by R¹³), phenyl (optionally substituted by halo or C₁₋₄alkyl), heteroaryl (optionally substituted by halo or C₁₋₄alkyl), heterocyclyl (optionally substituted by halo or C₁₋₄alkyl), C₁₋₄alkylthio, C₃₋₆cycloalkylthio, —SOR¹³, —SO₂R¹³, —SO₂NHR¹³, —SO₂NR¹³R¹⁴, —NHSO₂R¹³, —NR¹³SO₂R¹⁴, —NHCONHR¹³, —NHCONHR¹³R¹⁴, —OR¹³, cyano, —NR¹³R¹⁴, —CONR¹³R¹⁴ and —NHCOR¹³; or B is bicyclic aryl or heteroaryl where each is optionally substituted by one or more groups independently selected from nitro, trifluoromethyl, trifluoromethoxy, halo, C₁₋₄alkyl (optionally substituted by R¹³), C₂₋₄alkenyl (optionally substituted by R¹³), C₂₋₄alkynyl (optionally substituted by R¹³), C₃₋₆cycloalkyl (optionally substituted by R¹³), C₃₋₆cycloalkenyl (optionally substituted by R¹³), phenyl (optionally substituted by halo or C₁₋₄alkyl), heteroaryl (optionally substituted by halo or C₁₋₄alkyl), heterocyclyl (optionally substituted by halo or C₁₋₄alkyl), C₁₋₄alkylthio, C₃₋₆cycloalkylthio, —SOR¹³, —SO₂R¹³, —SO₂NHR¹³, —SO₂NR¹³R¹⁴, —NHSO₂R¹³, —NR¹³SO₂R¹⁴, —NHCONHR¹³, —NHCONHR¹³R¹⁴, —OR¹³, cyano, —NR¹³R¹⁴, —CONR¹³R¹⁴ and —NHCOR¹³;
R¹³ and R¹⁴ are independently hydrogen, C₁₋₆alkyl or C₃₋₆cycloalkyl;
or R¹³ and R¹⁴ together with the nitrogen to which they are attached form a heterocyclic 4 to 7-membered ring.

In a preferred embodiment of the invention:

Z is selected from —CONR¹⁵OH and —N(OH)CHO;

R¹⁵ is hydrogen or C₁₋₃alkyl;

R¹ is hydrogen or a group selected from C₁₋₆alkyl, C₂₋₆alkenyl, C₂₋₆alkynyl, C₃₋₇cycloalkyl, C₅₋₇cycloalkenyl, aryl and heteroaryl where the group is optionally substituted by one or more substituents independently selected from halo, nitro, cyano, trifluoromethyl, trifluoromethoxy, C₁₋₄alkyl, C₂₋₄alkenyl, C₂₋₄alkynyl, C₃₋₆cycloalkyl (optionally substituted by one or more R¹⁷), aryl (optionally substituted by one or more R¹⁷), heteroaryl (optionally substituted by one or more R¹⁷), heterocyclyl, C₁₋₄alkoxycarbonyl, —OR⁵, —SR², —SOR², —SO₂R², —COR², —CO₂R⁵, —CONR⁵R⁶, —NR¹⁶COR⁵, —SO₂NR⁵R⁶ and —NR¹⁶SO₂R²;

R¹⁶ is hydrogen or C₁₋₃alkyl;

R¹⁷ is selected from halo, C₁₋₆alkyl, C₃₋₆cycloalkyl and C₁₋₆alkoxy;

R² is group selected from C₁₋₆alkyl, C₃₋₆cycloalkyl, C₅₋₇cycloalkenyl, heterocycloalkyl, aryl, heteroaryl, arylC₁₋₄alkyl and heteroarylC₁₋₄alkyl where the group is optionally substituted by one or more halo;

R⁵ is hydrogen or a group selected from C₁₋₆alkyl, C₃₋₆cycloalkyl, C₅₋₇cycloalkenyl, heterocycloalkyl, aryl, heteroaryl, arylC₁₋₄alkyl and heteroarylC₁₋₄alkyl where the group is optionally substituted by one or more halo;

R⁶ is hydrogen, C₁₋₄alkyl or C₃₋₆cycloakyl;

or R⁵ and R⁶ together with the nitrogen to which they are attached form a heterocyclic 4 to 7-membered ring;

R⁸ is hydrogen or a group selected from C₁₋₆alkyl, C₃₋₇cycloalkyl and C₅₋₇cycloalkenyl where the group is optionally substituted by one or more substituents independently selected from halo, nitro, cyano, trifluoromethyl, trifluoromethoxy and C₁₋₄alkyl;

R³ and R⁴ are both hydrogen;

n is 0 or 1;

m is 0 or 1;

D is hydrogen, C₁₋₄alkyl, C₃₋₆cycloalkyl or fluoro;

X is O, S, SO or SO₂;

B is monocyclic aryl or heteroaryl where each is substituted in an ortho position by, and is optionally further substituted by, one or more groups independently selected from nitro, trifluoromethyl, trifluoromethoxy, halo, C₁₋₄alkyl (optionally substituted by R¹³), C₂₋₄alkenyl (optionally substituted by R¹³), C₂₋₄alkynyl (optionally substituted by R¹³), C₃₋₆cycloalkyl (optionally substituted by R¹³), C₃₋₆cycloalkenyl (optionally substituted by R¹³), phenyl (optionally substituted by halo or C₁₋₄alkyl), heteroaryl (optionally substituted by halo or C₁₋₄alkyl), heterocyclyl (optionally substituted by halo or C₁₋₄alkyl), C₁₋₄alkylthio, C₃₋₆cycloalkylthio, —SOR¹³, —SO₂R¹³, —SO₂NHR¹³, —SO₂NR¹³R¹⁴, —NHSO₂R¹³, —NR¹³SO₂R¹⁴, —NHCONH¹³, —NHCONHR¹³R¹⁴, —OR¹³, cyano, —CONR¹³R¹⁴, —NHCOR¹³, —CO²R¹³ and —CH₂CO₂R¹³;

or B is bicyclic aryl or heteroaryl where each is optionally substituted by one or more groups independently selected from nitro, trifluoromethyl, trifluoromethoxy, halo, C₁₋₄alkyl (optionally substituted by R¹³), C₂₋₄alkenyl (optionally substituted by R¹³), C₂₋₄alkynyl (optionally substituted by R¹³), C₃₋₆cycloalkyl (optionally substituted by R¹³), C₃₋₆cycloalkenyl (optionally substituted by R¹³), C₁₋₄alkylthio, C₃₋₆cycloalkylthio. —SOR¹³, —SO₂R¹³, —SO₂NHR¹³, —SO₂NR¹³R¹⁴, —NHSO₂R¹³, —NR¹³SO₂R¹⁴, —NHCONHR¹³, —NHCONHR¹³R¹⁴, —OR¹³, cyano, —CONR¹³R¹⁴ and —NHCOR¹³;

R¹³ and R¹⁴ are independently hydrogen, C₁₋₆alkyl or C₃₋₆cycloalkyl;

or R¹³ and R¹⁴ together with the nitrogen to which they are attached form a heterocyclic 4 to 7-membered ring.

Another aspect of the invention relates to compounds of formula (1) as hereinabove defined or to a pharmaceutically acceptable salt thereof.

It is to be understood that, insofar as certain of the compounds of formula (1) defined above may exist in optically active or racemic forms by virtue of one or more asymmetric carbon or sulphur atoms, the invention includes in its definition any such optically active or racemic form which possesses metalloproteinases inhibition activity and in particular TACE inhibition activity. The synthesis of optically active forms may be carried out by standard techniques of organic chemistry well known in the art, for example by synthesis from optically active starting materials or by resolution of a racemic form. Similarly, the above-mentioned activity may be evaluated using the standard laboratory techniques referred to hereinafter.

Compounds of formula (1) are therefore provided as enantiomers, diastereomers, geometric isomers and atropisomers.

Within the present invention it is to be understood that a compound of formula (1) or a salt thereof may exhibit the phenomenon of tautomerism and that the formulae drawings within this specification can represent only one of the possible tautomeric forms. It is to be understood that the invention encompasses any tautomeric form which has metalloproteinases inhibition activity and in particular TACE inhibition activity and is not to be limited merely to any one tautomeric form utilised within the formulae drawings. The formulae drawings within this specification can represent only one of the possible tautomeric forms and it is to be understood that the specification encompasses all possible tautomeric forms of the compounds drawn not just those forms which it has been possible to show graphically herein.

It is also to be understood that certain compounds of formula (1) and salts thereof can exist in solvated as well as unsolvated forms such as, for example, hydrated forms. It is to be understood that the invention encompasses all such solvated forms which have metalloproteinases inhibition activity and in particular TACE inhibition activity.

It is also to be understood that certain compounds of formula (1) may exhibit polymorphism, and that the invention encompasses all such forms which possess metalloproteinases inhibition activity and in particular TACE inhibition activity.

The present invention relates to the compounds of formula (1) as hereinbefore defined as well as to the salts thereof. Salts for use in pharmaceutical compositions will be pharmaceutically acceptable salts, but other salts may be useful in the production of the compounds of formula (1) and their pharmaceutically acceptable salts. Pharmaceutically acceptable salts of the invention may, for example, include acid addition salts of the compounds of formula (1) as hereinbefore defined which are sufficiently basic to form such salts. Such acid addition salts include but are not limited to hydrochloride, hydrobromide, citrate and maleate salts and salts formed with phosphoric and sulphuric acid. In addition where the compounds of formula (1) are sufficiently acidic, salts are base salts and examples include but are not limited to, an alkali metal salt for example sodium or potassium, an alkaline earth metal salt for example calcium or magnesium, or organic amine salt for example triethylamine or tris-(2-hydroxyethyl)amine.

The compounds of formula (1) may also be provided as in vivo hydrolysable esters. An in vivo hydrolysable ester of a compound of formula (1) containing carboxy or hydroxy group is, for example a pharmaceutically acceptable ester which is cleaved in the human or animal body to produce the parent acid or alcohol. Such esters can be identified by administering, for example, intravenously to a test animal, the compound under test and subsequently examining the test animal's body fluid.

Suitable pharmaceutically acceptable esters for carboxy include C₁₋₆alkoxymethyl esters for example methoxymethyl, C₁₋₆alkanoyloxymethyl esters for example pivaloyloxymethyl, phthalidyl esters, C₃₋₈cycloalkoxycarbonyloxyC₁₋₆alkyl esters for example 1-cyclohexylcarbonyloxyethyl; 1,3-dioxolen-2-onylmethyl esters for example 5-methyl-1,3-dioxolen-2-onylmethyl; and C₁₋₆alkoxycarbonyloxyethyl esters for example 1-methoxycarbonyloxyethyl and may be formed at any carboxy group in the compounds of this invention.

Suitable pharmaceutically-acceptable esters for hydroxy include inorganic esters such as phosphate esters (including phosphoramidic cyclic esters) and α-acyloxyalkyl ethers and related compounds which as a result of the Uh vivo hydrolysis of the ester breakdown to give the parent hydroxy group/s. Examples of α-acyloxyalkyl ethers include acetoxymethoxy and 2,2-dimethylpropionyloxymethoxy. A selection of in vivo hydrolysable ester forming groups for hydroxy include C₁₋₁₀alkanoyl, for example formyl, acetyl; benzoyl; phenylacetyl; substituted benzoyl and phenylacetyl, C₁₋₁₀alkoxycarbonyl (to give alkyl carbonate esters), for example ethoxycarbonyl; di-(C₁₋₄)alkylcarbamoyl and N-(di-(C₁₋₄)alkylaminoethyl)-N-(C₁₋₄)alkylcarbamoyl (to give carbamates); di-(C₁₋₄)alkylaminoacetyl and carboxyacetyl. Examples of ring substituents on phenylacetyl and benzoyl include aminomethyl, (C₁₋₄)alkylaminomethyl and di-((C₁₋₄)alkyl)aminomethyl, and morpholino or piperazino linked from a ring nitrogen atom via a methylene linking group to the 3- or 4-position of the benzoyl ring. Other interesting in vivo hydrolysable esters include, for example, R^{A}C(O)O(C₁₋₆)alkyl-CO—, wherein R^{A} is for example, benzyloxy-(C₁₋₄)alkyl, or phenyl). Suitable substituents on a phenyl group in such esters include, for example, 4-(C₁₋₄)piperazino-(C₁₋₄)alkyl, piperazino-(C₁₋₄)alkyl and morpholino-(C₁₋₄)alkyl.

In this specification the generic term “alkyl” includes both straight-chain and branched-chain alkyl groups. However references to individual alkyl groups such as “propyl” are specific for the straight chain version only and references to individual branched-chain alkyl groups such as tert-butyl are specific for the branched chain version only. For example, “C₁₋₃alkyl” includes methyl, ethyl, propyl and isopropyl, examples of “C₁₋₄alkyl” include the examples of “C₁₋₃alkyl”, butyl and tert-butyl and examples of “C₁₋₆alkyl” include the examples of “C₁₋₄alkyl” and additionally pentyl, 2,3-dimethylpropyl, 3-methylbutyl and hexyl. Examples of “C₁₋₂₀alkyl” include the examples of “C₁₋₆alkyl” and other straight-chain and branched chain alkyl groups. An analogous convention applies to other generic terms, for example “C₂₋₄alkenyl” includes vinyl, allyl and 1-propenyl and examples of “C₂₋₆alkenyl” include the examples of “C₂₋₄alkenyl” and additionally 1-butenyl, 2-butenyl, 3-butenyl, 2-methylbut-2-enyl, 3-methylbut-1-enyl, 1-pentenyl, 3-pentenyl and 4-hexenyl. Examples of “C₂₋₄alkynyl” includes ethynyl, 1-propynyl and 2-propynyl and examples of “C₂₋₆alkynyl” include the examples of “C₂₋₄alkynyl” and additionally 3-butynyl, 2-pentynyl and 1-methylpent-2-ynyl.

The term “C₃₋₆cycloalkyl” includes cyclopropyl, cyclobutyl, cyclopentyl and cyclohexyl. The term “C₃₋₇cycloalkyl” includes “C₃₋₆cycloalkyl” and additionally cycloheptyl. The term “C₃₋₁₀-cycloalkyl” includes “C₃₋₇cycloalkyl” and additionally cyclooctyl, cyclononyl and cyclodecyl.

“Heterocycloalkyl” is a monocyclic saturated 3- to 10-membered ring containing 1 or 2 heteroatoms selected from nitrogen, sulphur and oxygen wherein a ring nitrogen or sulphur may be oxidised to the N-oxide or S-oxide(s).

“C₅₋₇cycloalkenyl” is a monocyclic 5 to 7-member ring containing 1, 2 or 3 double bonds. Examples are cyclopentenyl and cyclohexenyl.

The term “halo” refers to fluoro, chloro, bromo and iodo.

Examples of “C₁₋₄alkoxy” include methoxy, ethoxy, propoxy and isopropoxy. Examples of “C₁₋₆alkoxy” include the examples of “C₁₋₄alkoxy” and additionally pentyloxy, 1-ethylpropoxy and hexyloxy. Examples of “C₁₋₄alkoxycarbonyl” include methoxycarbonyl, ethoxycarbonyl, propoxycarbonyl and isopropoxycarbonyl. Examples of “C₁₋₄alkylthio” include methylthio, ethylthio and propylthio. Examples of “C₃₋₆cycloalkylthio” include cyclopropylthio, cyclobutylthio and cyclopentylthio. Examples of “N—C₁₋₄alkylcarbamoyl” include methylcarbamoyl, ethylcarbamoyl, propylcarbamoyl, isopropylcarbamoyl and butylcarbamoyl. Examples of “N,N—(C₁₋₄alkyl)₂carbamoyl” include dimethylcarbamoyl, methyl(ethyl)carbamoyl and diethylcabamoyl.

Examples of “aryl” are phenyl and naphthyl. An example of “monocyclic aryl” is phenyl and an example of “bicyclic aryl” is naphthyl.

Examples of “arylC₁₋₄alkyl” are benzyl, phenylethyl, naphthylmethyl and naphthylethyl.

“Heteroaryl” is a monocyclic or bicyclic aryl ring containing 5 to 10 ring atoms of which 1, 2, 3 or 4 ring atoms are chosen from nitrogen, sulphur or oxygen where a ring nitrogen may be oxidised. Examples of heteroaryl are pyridyl, imidazolyl, quinolinyl, cinnolyl, pyrimidinyl, thienyl, pyrrolyl, pyrazolyl, thiazolyl, oxazolyl, isoxazolyl and pyrazinyl. Preferably heteroaryl is pyridyl, imidazolyl, quinolinyl, pyrimidinyl, thienyl, pyrazolyl, thiazolyl, oxazolyl and isoxazolyl. More preferably heteroaryl is pyridyl, pyrimidinyl, thienyl, quinolinyl, thieno[2,3-d]pyrimidinyl and thieno[3,2-d]pyrimidinyl. Examples of “monocyclic heteroaryl” are pyridyl, imidazolyl, pyrimidinyl, thienyl, pyrrolyl, pyrazolyl, thiazolyl, oxazolyl, isoxazolyl and pyrazinyl. Examples of “bicyclic heteroaryl” are quinolinyl, cinnolinyl, thieno[2,3-d]pyrimidinyl, thieno[3,2-d]pyrimidinyl and thieno[3,2-b]pyridyl.

Examples of “heteroarylC₁₋₄alkyl” are pyridylmethyl, pyridylethyl, pyrimidinylethyl, pyrimidinylpropyl, quinolinylpropyl and oxazolylmethyl.

“Heterocyclyl” is a saturated, partially saturated or unsaturated, monocyclic or bicycylic ring containing 4 to 12 atoms of which 1, 2, 3 or 4 ring atoms are chosen from nitrogen, sulphur or oxygen, which may, unless otherwise specified, be carbon or nitrogen linked, wherein a —CH₂— group can optionally be replaced by a —C(O)—, a ring nitrogen or sulphur atom may be optionally oxidised to form the N-oxide or S-oxide(s) and a —NH group may be optionally substituted by acetyl, formyl, methyl or mesyl. Examples and suitable values of the term “heterocyclyl” are piperidinyl, N-acetylpiperidinyl, N-methylpiperidinyl, piperazinyl, N-mesylpiperazinyl, N-formylpiperazinyl, homopiperazinyl, azetidinyl, oxetanyl, morpholinyl, tetrahydroisoquinolinyl, tetrahydroquinolinyl, indolinyl, pyranyl, dihydro-2H-pyranyl, tetrahydrofuranyl, 2,2-dimethyl-1,3-dioxolanyl and 3,4-dimethylenedioxybenzyl. Preferred values are 3,4-dihydro-2H-pyran-5-yl, tetrahydrofuran-2-yl, 2,2-dimethyl-1,3-dioxolan-2-yl and 3,4-dimethylenedioxybenzyl.

Heterocyclic rings are rings containing 1, 2 or 3 rings atoms selected nitrogen, oxygen and sulphur. “Heterocyclic 5 to 7-membered” rings are pyrrolidinyl, piperidinyl, piperizinyl, homopiperidinyl, homopiperazinyl, thiomorpholinyl, thiopyranyl and morpholinyl. “Heterocyclic 4 to 7-membered” rings include the examples of “heterocyclic 5 to 7-membered” and additionally azetidinyl.

“Saturated heterocyclic 3 to 6-membered” rings are oxiranyl, aziridinyl, thiirane, azetidinyl, oxetanyl, thietanyl, tetrahydrothienyl, pyrrolidinyl, tetrahydrofuranyl, tetrahydro-2H-pyranyl, tetrahydro-2H-thiopyranyl and piperidinyl and a ring nitrogen may be substituted by a group selected from formyl, acetyl and mesyl.

A “carbocyclic 3 to 6-membered” ring is a saturated, partially saturated or unsaturated ring containing 3 to 6 ring carbon atoms. Examples include cyclopropyl, cyclobutyl, cyclopentyl, cyclopent-3-enyl, cyclohexyl and cyclopent-2-enyl.

Where optional substituents are chosen from “one of more” groups or substituents it is to be understood that this definition includes all substituents being chosen from one of the specified groups or the substituents being chosen from two or more of the specified groups. Preferably “one or more” means “1, 2 or 3” and this is particularly the case when the group or substituent is halo. “One or more” may also means “1 or 2”.

Where monocyclic aryl or heteroaryl is substituted in “an ortho position” it is to be understood that the substituent is bonded to a ring atom which is immediately adjacent to the radical ring atom (wherein the radical ring atom is the ring atom bonded to X). For example an ortho substituent on pyrrol-2-yl would be located at position 1 (on the ring nitrogen) or position 3 (on a ring carbon). Similarly for pyrid-3-yl, an ortho substituent would be located at position 2 or position 4 (on a ring carbon) and for pyrid-2-yl, an ortho substituent would be located at position 3 (on a ring carbon). For phenyl ortho substituent would be located at position 2 or position 6.

Compounds of the present invention have been occasionally been named with the aid of computer software (ACD/Name version 5.09).

Preferred values of Z, R¹, R³, R⁴, R⁸, n, m, D, X and B are as follows. Such values may be used where appropriate with any of the definitions, claims or embodiments defined hereinbefore or hereinafter.

In one aspect of the present invention there is provided a compound of formula (1) as depicted above wherein Z is —CONR¹⁵OH. In another aspect of the invention Z is —N(OH)CHO.

In one aspect of the invention R¹⁵ is hydrogen, methyl, ethyl or isopropyl. In another aspect R¹⁵ is hydrogen. In a further aspect R¹⁵ is methyl, ethyl or isopropyl.

In one aspect of the invention R¹ is hydrogen or a group selected from C₁₋₆alkyl, C₂₋₆alkynyl, C₃₋₇cycloalkyl, aryl and heteroaryl where the group is optionally substituted by one or more substituents independently selected from halo, nitro, cyano, trifluoromethyl, C₁₋₄alkyl, C₃₋₆-cycloalkyl, aryl (optionally substituted by R¹⁷), heteroaryl (optionally substituted by R¹⁷), C₁₋₄alkoxycarbonyl, —OR⁵, —SR², —SOR², —SO₂R², —COR², —CO₂R⁵ and —NR¹⁶COR⁵. In another aspect R¹ is a group selected from C₁₋₆alkyl, aryl and heteroaryl each being optionally substituted by one or more substituents independently selected from C₁₋₆alkyl, C₃₋₆cycloalkyl, (optionally substituted by R¹⁷), aryl (optionally substituted by R¹⁷) and heteroaryl (optionally substituted by R¹⁷). In another aspect R¹ is a group selected from C₁₋₆alkyl, C₃₋₆cycloalkyl, aryl, heteroaryl and C₁₋₄alkyl substituted by aryl or heteroaryl wherein any R¹ group is optionally substituted by one or more substituents independently selected from halo, C₁₋₄alkoxy, C₁₋₄alkyl and C₃₋₆cycloalkyl. In another aspect of the invention R¹ is hydrogen or a group selected from methyl, ethyl, propyl, isopropyl, tert-butyl, isobutyl, ethynyl, cyclopropyl, cyclobutyl, cyclopentyl, cyclohexyl, phenyl, naphthyl, pyridyl, thienyl, pyrimidinyl, quinolinyl, thiazolyl, oxazolyl, isoxazolyl, pyrazolyl and imidazolyl, where the group is optionally substituted by one or more substituents independently selected from fluoro, chloro, bromo, nitro, cyano, trifluoromethyl, methyl, ethyl, C₃₋₆cycloalkyl, phenyl (optionally substituted by halo or C₁₋₄alkyl), pyrimidinyl (optionally substituted by halo or C₁₋₄alkyl), C₁₋₄alkoxycarbonyl, —OR⁵, —SR², —SOR², —SO₂R², —COR², —CO₂R⁵ and —NR¹⁶COR⁵. In a further aspect of the invention R¹ is selected from hydrogen, methyl, ethyl, propyl, isopropyl, tert-butyl, isobutyl, cyclopropyl, cyclobutyl, cyclopentyl, cyclohexyl, benzyloxymethyl, phenyl, benzyl, phenylethyl, phenylpropyl, (5-fluoropyrimidin-2-yl)ethyl, (5-fluoropyrimidin-2-yl)propyl, pyrimidin-2-ylethyl, pyrimidin-2-ylpropyl, naphth-2-yl, naphth-1-yl, 3,4-dichlorophenyl, 4-chlorophenyl, biphenylyl, 3-nitrophenyl, 2-trifluoromethylphenyl, 3-trifluoromethylphenyl, 4-trifluoromethylphenyl, 3-bromophenyl, 4-(methoxycarbonyl)phenyl, 4-benzyloxyphenyl, 2-fluorophenyl, 3-fluorophenyl, 4-fluorophenyl, 3-(4-chlorophenoxy)phenyl, 2-cyanophenyl, 3-cyanophenyl, 4-cyanophenyl, 2-bromothien-5-yl, 2-methylthien-5-yl, pyrimidin-2-yl, 2-methylpyrimidin-5-yl, 2-methylpyrimidin-4-yl, quinolin-4-yl, ethynyl, methoxymethyl, thiazol-2-yl, oxazol-2-yl, isoxazol-5-yl, 4,4-difluorocyclohexyl, pyrimidin-2-ylmethyl, 2-pyrimidin-2-ylethyl, 3-pyrimidin-2-ylpropyl, 2,2,2-trifluoroethyl, 3-bromo-4-hydroxyphenyl, 4-fluoro-2-trifluoromethylphenyl, pyrid-2-yl, pyrid-3-yl, pyrid-4-yl, imidazol-4-yl, 1H-imidazol-4-yl, pyrazol-3-yl, 1H-pyrazol-3-yl and (N-acetylamino)phenyl. In another aspect R¹ is propyl, cyclopentyl, phenyl or pyridyl optionally substituted by methyl, ethyl, phenyl, pyridyl or pyrimidinyl. In a further aspect R¹ is isobutyl, cyclopentyl, 3-(pyrimindin-2-yl)propyl, phenyl or pyrid-3-yl.

In one aspect of the invention R¹⁶ is hydrogen, methyl or ethyl. In another aspect R¹⁶ is methyl or ethyl. In another aspect of the invention R¹⁶ is hydrogen.

In one aspect of the invention R¹⁷ is halo or C₁₋₄alkyl. In another aspect R¹⁷ is fluoro, chloro, bromo or methyl. In another aspect of the invention R¹⁷ is fluoro or methyl.

In one aspect of the invention R² is a group selected from C₁₋₆alkyl, aryl and arylC₁₋₄alkyl where the group is optionally substituted by halo. In another aspect R² is a group selected from methyl, phenyl and benzyl where the group is optionally substituted by chloro. In one aspect of the invention R² is methyl.

In one aspect of the invention R⁵ is hydrogen or a group selected from C₁₋₆alkyl, aryl and arylC₁₋₄alkyl where the group is optionally substituted by halo. In another aspect R⁵ is hydrogen or a group selected from methyl, phenyl and benzyl where the group is optionally substituted by chloro.

In one aspect of the invention R⁶ is hydrogen, methyl, ethyl, propyl or isopropyl.

In one aspect of the invention R⁸ is hydrogen, methyl, ethyl, propyl or isopropyl. In another aspect R⁸ is hydrogen.

In one aspect of the invention R³ is hydrogen, methyl, ethyl or phenyl. In another aspect R³ is hydrogen.

In one aspect of the invention R⁴ is hydrogen, methyl, ethyl or phenyl. In another aspect R⁴ is hydrogen.

In one aspect of the invention n is 0. In another aspect n is 1.

In one aspect of the invention m is 0. In another aspect of the invention m is 1.

In one aspect of the invention D is hydrogen, methyl or fluoro. In another aspect D is hydrogen.

In one aspect of the invention X is O.

In one aspect of the invention B is phenyl or pyridyl where each is substituted in an ortho position by, and is optionally further substituted by one or more groups independently selected from chloro, fluoro, bromo, trifluoromethyl, cyano, acetamido, propyloxy, methoxy, methyl, nitro, pyrrolidinylcarbonyl, N-propylcarbamoyl, pyrrolidinyl, piperidinyl, isoxazolyl, pyrazolyl, imidazolyl, oxazolyl, thiazolyl, pyrimidinyl and pyridyl; or B is naphthyl, quinolinyl, 1,6-naphthyridinyl, thieno[2,3-d]pyrimidinyl, thieno[3,2-d]pyrimidinyl or thieno[3,2-b]pyridyl each being optionally substituted by one or more groups independently selected from chloro, fluoro, bromo, trifluoromethyl, cyano, acetamido, propyloxy, methoxy, methyl, nitro, pyrrolidinylcarbonyl N-propylcarbamoyl, pyrrolidinyl, piperidinyl, isoxazolyl, pyrazolyl, imidazolyl, oxazolyl, thiazolyl, pyrimidinyl and pyridyl. In one aspect of the invention B is phenyl or pyridyl where each is substituted in an ortho position by, and is optionally further substituted by one or more groups independently selected from chloro, fluoro, bromo, trifluoromethyl, cyano, acetamido, propyloxy, methoxy, ethoxy, isopropoxy, methyl, ethyl, propyl, isopropyl, nitro, pyrrolidinylcarbonyl, N-propylcarbamoyl, N-isopropylcarbamoyl, N-ethylcarbamoyl and N-methylcarbamoyl; or B is naphthyl, quinolinyl, 1,6-naphthyridinyl, thieno[2,3-d]pyrimidinyl, thieno[3,2-d]pyrimidinyl or thieno[3,2-b]pyridyl each being optionally substituted by one or more groups independently selected from chloro, fluoro, bromo, trifluoromethyl, cyano, acetamido, propyloxy, methoxy, methyl, nitro, pyrrolidinylcarbonyl and N-propylcarbamoyl. In another aspect B is phenyl or pyridyl where each is substituted in an ortho position by, and is optionally further substituted by one or more groups independently selected from halo, trifluoromethyl, cyano, C₁₋₄alkoxy, C₁₋₄alkyl, nitro, aryl, heteroaryl, heterocyclyl, N—(C₁₋₄alkyl)carbamoyl and N,N—(C₁₋₄alkyl)₂carbamoyl; or B is naphthyl, quinolinyl, thieno[2,3-d]pyrimidinyl or thieno[3,2-d]pyrimidinyl each being optionally substituted by one or more groups independently selected from halo, trifluoromethyl, cyano, C₁₋₄alkoxy, C₁₋₄alkyl, aryl, heteroaryl, heterocyclyl and nitro. In another aspect B is phenyl or pyridyl where each is substituted in an ortho position by, and is optionally further substituted by one or more groups independently selected from halo, trifluoromethyl, cyano, C₁₋₄alkoxy, C₁₋₄alkyl, nitro, N—(C₁₋₄alkyl)carbamoyl and N,N—(C₁₋₄alkyl)₂carbamoyl; or B is naphthyl, quinolinyl, thieno[2,3-d]pyrimidinyl or thieno[3,2-d]pyrimidinyl all being optionally substituted by one or more groups independently selected from halo, trifluoromethyl, cyano, C₁₋₄alkoxy, C₁₋₄alkyl and nitro. In one aspect of the invention B is phenyl or pyridyl where each is substituted in an ortho position by, and is optionally further substituted by one or more groups independently selected from chloro, fluoro, bromo, trifluoromethyl, cyano, isopropyloxy, methoxy, methyl, nitro, N-isopropylcarbamoyl, phenyl, pyridyl, pyrimidinyl, thienyl, isoxazolyl and piperidinyl; or B is naphthyl, thieno[2,3-d]pyrimidinyl or thieno[3,2-d]pyrimidinyl each being optionally substituted by one or more groups independently selected from chloro, fluoro, bromo, trifluoromethyl, cyano, methoxy, methyl, nitro, phenyl, pyridyl, pyrimidinyl, thienyl, isoxazolyl and piperidinyl. In one aspect of the invention B is phenyl or pyridyl where each is substituted in an ortho position by, and is optionally further substituted by one or more groups independently selected from chloro, fluoro, bromo, trifluoromethyl, cyano, isopropyloxy, methoxy, methyl, nitro and N-isopropylcarbamoyl; or B is naphthyl, thieno[2,3-d]pyrimidinyl or thieno[3,2-d]pyrimidinyl each being optionally substituted by one or more groups independently selected from chloro, fluoro, bromo, trifluoromethyl, cyano, methoxy, methyl and nitro. In another aspect B is selected from naphthyl, 2-chloro-4-fluorophenyl, 2-chloro-4-trifluoromethylphenyl, 2-bromo-4,6-difluorophenyl, 2-bromo-4-fluorophenyl, 2,4-dichlorophenyl, 2-cyanophenyl, 2-bromophenyl, 2-chlorophenyl, 2-acetamidophenyl, 2-(isopropyloxy)phenyl, 2-trifluoromethylphenyl, 2-bromo-4-chlorophenyl, 2-methoxy-4-methylphenyl, 4-chloro-2-nitrophenyl, 4-methyl-2-nitrophenyl, 2,4-difluorophenyl, 2-nitrophenyl, 4-bromo-2-fluorophenyl, 2-methoxy-4-nitrophenyl, 2-(pyrrolidin-1-ylcarbonyl)phenyl, 2-chloro-4-nitrophenyl, 2-(N-isopropyl)carbamoylphenyl, 2-(pyrrolidin-1-yl)phenyl, 2-(piperidin-1-yl)phenyl, 4-bromo-2-methoxyphenyl, 2-fluoro-4-nitrophenyl, 2-chloro-4-bromophenyl, 2-chloro-4-methylphenyl, 2-chloro-4-methoxyphenyl, 4-fluoro-2-methoxyphenyl, 2-fluoro-4-chlorophenyl, 4-fluoro-2-methylphenyl, 2-(isoxazol-5-yl)phenyl, 3-chloropyrid-2-yl, quinolin-4-yl, 7-chloroquinolinyl, 3-cyanopyrid-2-yl, 8-chloroquinolin-4-yl, 3-trifluoromethylpyrid-2-yl, 3-chloro-5-trifluoromethylpyrid-2-yl, 3,5-dichloropyrid-2-yl, 6-chloroquinolin-4-yl, 5-methylthieno[2,3-d]pyrimidin-4-yl, 7-methylthieno[3,2-d]pyrimidin-4-yl, 8-fluoroquinolin-4-yl, 2-pyrazol-5-ylphenyl, 4-chloro-2-(isoxazol-5-yl)phenyl, 2-(isoxazol-5-yl)-4-trifluoromethylphenyl, 2-imidazol-5-ylphenyl, 2-(oxazol-5-yl)phenyl, 2-(thiazol-5-yl)phenyl, 2-(pyrimidin-2-yl)phenyl, 2-(pyrid-2-yl)phenyl, 6-fluoroquinolin-4-yl, 2-methylquinolin-4-yl, 6-chloro-2-methylquinolin-4-yl, 1,6-naphthyridin-4-yl, thieno[3,2-b]pyrid-7-yl, 5-fluoro-2-(isoxazol-5-yl)phenyl, 4-fluoro-2-(isoxazol-5-yl)phenyl, 4-chloro-2-trifluoromethylphenyl and 2-chloro-5-fluorophenyl. In another aspect B is 4-fluoro-2-thiophenyl)phenyl, 4-fluoro-2-(pyrid-2-yl)phenyl. In a further aspect B is selected from 2-chloro-4-trifluoromethylphenyl, 2-bromo-4-fluorophenyl, 2-bromophenyl, 2-chlorophenyl, 2-chloro-4-fluorophenyl, 2,4-dichlorophenyl, 2-(isopropyloxy)phenyl, 2-trifluoromethylphenyl, 4-chloro-2-nitrophenyl, 4-methyl-2-nitrophenyl, 2-methoxy-4-nitrophenyl, 2-(N-isopropyl)carbamoylphenyl, 2-fluoro-4-nitrophenyl, 2-chloro-4-methylphenyl, 2-chloro-4-methoxyphenyl, 4-fluoro-2-methoxyphenyl, 2-fluoro-4-chlorophenyl, 4-fluoro-2-methylphenyl, 3-chloropyrid-2-yl, 3-cyanopyrid-2-yl, 8-chloroquinolin-4-yl, 3-trifluoromethylpyrid-2-yl, 3-chloro-5-trifluoromethylpyrid-2-yl, 3,5-dichloropyrid-2-yl, 5-methylthieno[2,3-d]pyrimidin-4-yl, 7-methylthieno[3,2-d]pyrimidin-4-yl, naphthyl, 2-bromo-4,6-difluorophenyl, 2-cyanophenyl, 2-isoxazol-5-ylphenyl, 2-piperidin-1-ylphenyl, 4-fluoro-2-thien-3-ylphenyl and 4-fluoro-2-pyrid-3-ylphenyl. In a further aspect B is selected from 2-chloro-4-trifluoromethylphenyl, 2-bromo-4-fluorophenyl, 2-bromophenyl, 2-chlorophenyl, 2-chloro-4-fluorophenyl, 2,4-dichlorophenyl, 2-(isopropyloxy)phenyl, 2-trifluoromethylphenyl, 4-chloro-2-nitrophenyl, 4-methyl-2-nitrophenyl, 2-methoxy-4-nitrophenyl, 2-(N-isopropyl)carbamoylphenyl, 2-fluoro-4-nitrophenyl, 2-chloro-4-methylphenyl, 2-chloro-4-methoxyphenyl, 4-fluoro-2-methoxyphenyl, 2-fluoro-4-chlorophenyl, 4-fluoro-2-methylphenyl, 3-chloropyrid-2-yl, 3-cyanopyrid-2-yl, 8-chloroquinolin-4-yl, 3-trifluoromethylpyrid-2-yl, 3-chloro-5-trifluoromethylpyrid-2-yl, 3,5-dichloropyrid-2-yl, 5-methylthieno[2,3-d]pyrimidin-4-yl, 7-methylthieno[3,2-d]pyrimidin-4-yl, naphthyl, 2-bromo-4,6-difluorophenyl and 2-cyanophenyl.

In one aspect of the invention R¹³ is C₁₋₆alkyl. In another aspect R¹³ is methyl or isopropyl.

In one aspect of the invention R¹⁴ is hydrogen

In one aspect of the invention R¹³ and R¹⁴ together with the nitrogen to which they are attached form pyrrolidinyl or piperidinyl.

A preferred class of compound is of formula (I) wherein;

Z is —N(OH)CHO;

R¹ is hydrogen or a group selected from C₁₋₆alkyl, C₂₋₆alkynyl, C₃₋₇cycloalkyl, aryl and heteroaryl where the group is optionally substituted by one or more substituents independently selected from halo, nitro, cyano, trifluoromethyl, C₁₋₄alkyl, C₃₋₆cycloalkyl, aryl (optionally substituted by R¹⁷), heteroaryl (optionally substituted by R¹⁷), C₁₋₄alkoxycarbonyl, —OR⁵, —SR², —SOR², —SO₂R², —COR², —CO₂R⁵ and —NR¹⁶COR⁵;

R¹⁶ is hydrogen, methyl or ethyl;

R¹⁷ is halo or C₁₋₄alkyl;

R² is a group selected from C₁₋₆alkyl, aryl and arylC₁₋₄alkyl where the group is optionally substituted by halo;

R⁵ is hydrogen or a group selected from C₁₋₆alkyl, aryl and arylC₁₋₄alkyl where the group is optionally substituted by halo;

R⁶ is hydrogen, methyl, ethyl, propyl or isopropyl;

R⁸ is hydrogen, methyl, ethyl, propyl or isopropyl;

R³ is hydrogen;

R⁴ is hydrogen;

n is 0;

m is 1;

D is hydrogen, methyl or fluoro;

X is O;

B is phenyl or pyridyl where each is substituted in an ortho position by, and is optionally further substituted by one or more groups independently selected from halo, trifluoromethyl, cyano, C₁₋₄alkoxy, C₁₋₄alkyl, nitro, aryl, heteroaryl, heterocyclyl, N—(C₁₋₄alkyl)carbamoyl and N,N—(C₁₋₄alkyl)₂carbamoyl; or B is naphthyl, quinolinyl, thieno[2,3-d]pyrimidinyl or thieno[3,2-d]pyrimidinyl all being optionally substituted by one or more groups independently selected from halo, trifluoromethyl, cyano, C₁₋₄alkoxy, C₁₋₄alkyl, aryl, heteroaryl, heterocyclyl and nitro.

Another preferred class of compound is of formula (1) wherein:

Z is —CONR¹⁵(OH);

R¹⁵ is hydrogen, methyl, ethyl or isopropyl

R¹ is hydrogen or a group selected from C₁₋₆alkyl, C₂₋₆alkynyl, C₃₋₇cycloalkyl, aryl and heteroaryl where the group is optionally substituted by one or more substituents independently selected from halo, nitro, cyano, trifluoromethyl, C₁₋₄alkyl, C₃₋₆cycloalkyl, aryl (optionally substituted by R¹⁷), heteroaryl (optionally substituted by R¹⁷), C₁₋₄alkoxycarbonyl, —OR⁵, —SR², —SOR², —SO₂R², —COR², —CO₂R⁵ and —NR¹⁶COR⁵;

R¹⁶ is hydrogen, methyl or ethyl;

R¹⁷ is halo or C₁₋₄alkyl;

R² is a group selected from C₁₋₆alkyl, aryl and arylC₁₋₄alkyl where the group is optionally substituted by halo;

R⁵ is hydrogen or a group selected from C₁₋₆alkyl, aryl and arylC₁₋₄alkyl where the group is optionally substituted by halo;

R³ is hydrogen;

R⁴ is hydrogen;

R⁸ is hydrogen;

n is 0;

m is 1;

D is hydrogen, methyl or fluoro;

X is O;

B is phenyl or pyridyl where each is substituted in an ortho position by, and is optionally further substituted by one or more groups independently selected from halo, trifluoromethyl, cyano, C₁₋₄alkoxy, C₁₋₄alkyl, nitro, aryl, heteroaryl, heterocyclyl, N—(C₁₋₄alkyl)carbamoyl and N,N—(C₁₋₄alkyl)₂carbamoyl; or B is naphthyl, quinolinyl, thieno[2,3-d]pyrimidinyl or thieno[3,2-d]pyrimidinyl all being optionally substituted by one or more groups independently selected from halo, trifluoromethyl, cyano, C₁₋₄alkoxy, C₁₋₄alkyl, aryl, heteroaryl, heterocyclyl and nitro.

Another preferred class is of compound of formula (1) wherein:

Z is —CONHOH or —N(OH)CHO;

R¹ is a group selected from C₁₋₆alkyl, C₃₋₆cycloalkyl, aryl, heteroaryl and C₁₋₆alkyl substituted by aryl or heteroaryl wherein any R¹ group is optionally substituted by one or more substituents independently selected from halo, C₁₋₄alkoxy, C₁₋₄alkyl and C₃₋₆cycloalkyl;

R³ is hydrogen;

R⁴ is hydrogen;

R⁸ is hydrogen;

n is 0;

m is 1;

D is hydrogen;

X is O; and

B is phenyl or pyridyl where each is substituted in an ortho position by, and is optionally further substituted by one or more groups independently selected from halo, trifluoromethyl, cyano, C₁₋₄alkoxy, C₁₋₄alkyl, nitro, N—(C₁₋₄alkyl)carbamoyl and N,N—(C₁₋₄alkyl)₂carbamoyl; or B is naphthyl, quinolinyl, thieno[2,3-d]pyrimidinyl or thieno[3,2-d]pyrimidinyl all being optionally substituted by one or more groups independently selected from halo, trifluoromethyl, cyano, C₁₋₄alkoxy, C₁₋₄alkyl and nitro.

Another preferred class is of compound of formula (1) wherein:

Z is —CONHOH or N(OH)CHO;

R¹ is a group selected from C₁₋₆alkyl, C₃₋₆cycloalkyl, aryl, heteroaryl and C₁₋₆alkyl substituted by aryl or heteroaryl wherein any R¹ group is optionally substituted by one or more substituents independently selected from halo, C₁₋₄alkoxy, C₁₋₄alkyl and C₃₋₆cycloalkyl;

R³ is hydrogen;

R⁴ is hydrogen;

R⁸ is hydrogen;

n is 0;

m is 1;

D is hydrogen;

B is phenyl or pyridyl where each is substituted in an ortho position by, and is optionally further substituted by one or more groups independently selected from chloro, fluoro, bromo, trifluoromethyl, cyano, isopropyloxy, methoxy, methyl, nitro, N-isopropylcarbamoyl, phenyl pyridyl, pyrimidinyl, thienyl, isoxazolyl and piperidinyl; or B is naphthyl, thieno[2,3-d]pyrimidinyl or thieno[3,2-d]pyrimidinyl each being optionally substituted by one or more groups independently selected from chloro, fluoro, bromo, trifluoromethyl, cyano, methoxy, methyl, nitro, phenyl, pyridyl, pyrimidinyl, thienyl, isoxazolyl and piperidinyl.

Another preferred class is of compound of formula (1) wherein:

Z is —CONHOH or —N(OH)CHO;

R¹ is propyl, cyclopentyl, phenyl or pyridyl optionally substituted by methyl, ethyl, phenyl, pyridyl or pyrimidinyl;

R³ is hydrogen;

R⁴ is hydrogen;

R⁸ is hydrogen;

n is 0;

m is 1;

D is hydrogen;

X is O; and

B is phenyl or pyridyl where each is substituted in an ortho position by, and is optionally further substituted by one or more groups independently selected from chloro, fluoro, bromo, trifluoromethyl, cyano, isopropyloxy, methoxy, methyl, nitro and N-isopropylcarbamoyl; or B is naphthyl, thieno[2,3-d]pyrimidinyl or thieno[3,2-d]pyrimidinyl each being optionally substituted by one or more groups independently selected from chloro, fluoro, bromo, trifluoromethyl, cyano, methoxy, methyl and nitro.

In another aspect of the invention, preferred compounds of the invention are any one of:
1-({[4-(1-naphthyloxy)piperidin-1-yl]sulphonyl}methyl)-4-pyrimidin-2-ylbutyl(hydroxy)formamide;
1-({[4-(2-chloro-4-fluorophenoxy)piperidin-1-yl]sulphonyl}methyl)-4-pyrimidin-2-ylbutyl(hydroxy)formamide;
1-[({4-[2-chloro-4-(trifluoromethyl)phenoxy]piperidin-1-yl}sulphonyl)methyl]-4-pyrimidin-2-ylbutyl(hydroxy)formamide;
1-({[4-(2-bromo-4,6-difluorophenoxy)piperidin-1-yl]sulphonyl}methyl)-4-pyrimidin-2-ylbutyl(hydroxy)formamide;
1-({[4-(2-bromo-4-fluorophenoxy)piperidin-1-yl]sulphonyl}methyl)-4-pyrimidin-2-ylbutyl(hydroxy)formamide;
1-({[4-(2,4-dichlorophenoxy)piperidin-1-yl]sulphonyl}methyl)-4-pyrimidin-2-ylbutyl(hydroxy)formamide;
1-({[4-(2-cyanophenoxy)piperidin-1-yl]sulphonyl}methyl)-4-pyrimidin-2-ylbutyl(hydroxy)formamide;
2-{[4-(2-cyanophenoxy)piperidin-1-yl]sulphonyl}-1-phenylethyl(hydroxy)formamide;
2-{[4-(2-bromophenoxy)piperidin-1-yl]sulphonyl}-1-phenylethyl(hydroxy)formamide;
2-{[4-(2-chlorophenoxy)piperidin-1-yl]sulphonyl}-1-phenylethyl(hydroxy)formamide;
2-{[4-(2-chloro-4-fluorophenoxy)piperidin-1-yl]sulphonyl}-1-phenylethyl(hydroxy)formamide;
2-{[4-(2,4-dichlorophenoxy)piperidin-1-yl]sulphonyl}-1-phenylethyl(hydroxy)formamide;
2-{[4-(2-acetamidophenoxy)piperidin-1-yl]sulphonyl}-1-phenylethyl(hydroxy)formamide;
2-{[4-(2-isopropoxyphenoxy)piperidin-1-yl]sulphonyl}-1-phenylethyl(hydroxy)formamide;
2-({4-[2-(trifluoromethyl)phenoxy]piperidin-1-yl}sulphonyl)-1-phenylethyl(hydroxy)formamide;
2-{[4-(2-bromo-4-chlorophenoxy)piperidin-1-yl]sulphonyl}-1-phenylethyl(hydroxy)formamide;
2-{[4-(2-methoxy-4-methylphenoxy)piperidin-1-yl]sulphonyl}-1-phenylethyl(hydroxy)formamide;
2-{[4-(4-chloro-2-nitrophenoxy)piperidin-1-yl]sulphonyl}-1-phenylethyl(hydroxy)formamide;
2-{[4-(4-methyl-2-nitrophenoxy)piperidin-1-yl]sulphonyl}-1-phenylethyl(hydroxy)formamide;
2-{[4-(2,4-difluorophenoxy)piperidin-1-yl]sulphonyl}-1-phenylethyl(hydroxy)formamide;
2-{[4-(2-bromo-4-fluorophenoxy)piperidin-1-yl]sulphonyl}-1-phenylethyl(hydroxy)formamide;
2-{[4-(2-nitrophenoxy)piperidin-1-yl]sulphonyl}-1-phenylethyl(hydroxy)formamide;
2-{[4-(4-bromo-2-fluorophenoxy)piperidin-1-yl]sulphonyl}-1-phenylethyl(hydroxy)formamide;
2-{[4-(2-methoxy-4-nitrophenoxy)piperidin-1-yl]sulphonyl}-1-phenylethyl(hydroxy)formamide;
2-({4-[2-(pyrrolidin-1-ylcarbonyl)phenoxy]piperidin-1-yl}sulphonyl)1-phenylethyl(hydroxy)formamide;
2-{[4-(2-chloro-4-nitrophenoxy)piperidin-1-yl]sulphonyl}-1-phenylethyl(hydroxy)formamide;
2-{[4-(2-(N-isopropylcarbamoyl)phenoxy)piperidin-1-yl]sulphonyl}-1-phenylethyl(hydroxy)formamide;
2-{[4-(2-pyrrolidin-1-ylphenoxy)piperidin-1-yl]sulphonyl}-1-phenylethyl(hydroxy)formamide;
2-{[4-(2-piperidin-1-ylphenoxy)piperidin-1-yl]sulphonyl}-1-phenylethyl(hydroxy)formamide;
2-{[4-(4-bromo-2-methoxyphenoxy)piperidin-1-yl]sulphonyl}-1-phenylethyl(hydroxy)formamide;
2-{[4-(2-fluoro-4-nitrophenoxy)piperidin-1-yl]sulphonyl}-1-phenylethyl(hydroxy)formamide;
2-{[4-(2-chloro-4-methylphenoxy)piperidin-1-yl]sulphonyl}-1-phenylethyl(hydroxy)formamide;
2-{[4-(2-chloro-4-methoxyphenoxy)piperidin-1-yl]sulphonyl}-1-phenylethyl(hydroxy)formamide;
2-{[4-(4-fluoro-2-methoxyphenoxy)piperidin-1-yl]sulphonyl}-1-phenylethyl(hydroxy)formamide;
2-{[4-(4-chloro-2-fluorophenoxy)piperidin-1-yl]sulphonyl}-1-phenylethyl(hydroxy)formamide;
2-{[4-(4-fluoro-2-methylphenoxy)piperidin-1-yl]sulphonyl}-1-phenylethyl(hydroxy)formamide;
2-{[4-(2-isoxazol-5-ylphenoxy)piperidin-1-yl]sulphonyl}-1-phenylethyl(hydroxy)formamide;
2-({4-[(3-chloropyrid-2-yl)oxy]piperidin-1-yl}sulphonyl)-1-pyrid-3-ylethyl(hydroxy)formamide;
2-{[4-(quinolin-4-yloxy)piperidin-1-yl]sulphonyl}-1-pyrid-3-ylethyl(hydroxy))formamide;
2-({4-[(7-chloroquinolin-4-yl)oxy]piperidin-1-yl}sulphonyl)-1-pyrid-3-ylethyl(hydroxy)formamide;
2-({4-[(3-cyanopyrid-2-yl)oxy]piperidin-1-yl}sulphonyl)-1-pyrid-3-ylethyl(hydroxy)formamide;
2-({4-[(8-chloroquinolin-4-yl)oxy]piperidin-1-yl}sulphonyl)-1-pyrid-3-ylethyl(hydroxy)formamide;
2-[(4-{[3-(trifluoromethyl)pyrid-2-yl]oxy}piperidin-1-yl)sulphonyl]-1-pyrid-3-yl ethyl(hydroxy)formamide;
2-[(4-{[3-chloro-5-(trifluoromethyl)pyrid-2-yl]oxy}piperidin-1-yl)sulphonyl]-1-pyrid-3-ylethyl(hydroxy)formamide;
2-({4-[(3,5-dichloropyrid-2-yl)oxy]piperidin-1-yl}sulphonyl)-1-pyrid-3-ylethyl(hydroxy)formamide;
2-({4-[(6-chloroquinolin-4-yl)oxy]piperidin-1-yl}sulphonyl)-1-pyrid-3-ylethyl(hydroxy)formamide;
2-({4-[(5-methylthieno[2,3-d]pyrimidin-4-yl)oxy]piperidin-1-yl}sulphonyl)-1-pyrid-3-ylethyl(hydroxy)formamide;
2-({4-[(7-methylthieno[3,2-d]pyrimidin-4-yl)oxy]piperidin-1-yl}sulphonyl)-1-pyrid-3-ylethyl(hydroxy)formamide; and
2-({4-[(8-fluoroquinolin-4-yl)oxy]piperidin-1-yl}sulphonyl)-1-pyrid-3-ylethyl(hydroxy)formamide.

In a further aspect of the invention, preferred compounds of the invention are any one of:
(R/S)-1-[({4-[2-chloro-4-(trifluoromethyl)phenoxy]piperidin-1-yl}sulphonyl)methyl]-4-pyrimidin-2-ylbutyl(hydroxy)formamide;
(R/S)-1-({[4-(2-bromo-4-fluorophenoxy)piperidin-1-yl]sulphonyl}methyl)-4-pyrimidin-2-ylbutyl(hydroxy)formamide;
(R/S)-2-{[4-(2-bromophenoxy)piperidin-1-yl]sulphonyl}-1-phenylethyl(hydroxy)formamide;
(R/S)-2-{[4-(2-chlorophenoxy)piperidin-1-yl]sulphonyl}-1-phenylethyl(hydroxy)formamide;
(R/S)-2-{[4-(2-chloro-4-fluorophenoxy)piperidin-1-yl]sulphonyl}-1-phenylethyl(hydroxy)formamide;
(R/S)-2-{[4-(2,4-dichlorophenoxy)piperidin-1-yl]sulphonyl}-1-phenylethyl(hydroxy)formamide;
(R/S)-hydroxy(2-{[4-(2-isopropoxyphenoxy)piperidin-1-yl]sulphonyl}-1-phenylethyl)formamide;
(R/S)-hydroxy(2-{[4-(2-trifluoromethylphenoxy)piperidin-1-yl]sulphonyl}-1-phenylethyl)formamide;
(R/S)-2-{[4-(4-chloro-2-nitrophenoxy)piperidin-1-yl]sulphonyl}-1-phenylethyl(hydroxy)formamide;
(R/S)-hydroxy(2-[{4-(4-methyl-2-nitrophenoxy)piperidin-1-yl]sulphonyl}-1-phenylethyl)formamide;
(R/S)-2-{[4-(2-bromo-4-fluorophenoxy)piperidin-1-yl]sulphonyl}-1-phenylethyl(hydroxy)formamide;
(R/S)-hydroxy(2-{[4-(2-methoxy-4-nitrophenoxy)piperidin-1-yl]sulphonyl}-1-phenylethyl)formamide;
(R/S)-hydroxy[2-({4-[2-(isopropylaminocarbonyl)phenoxy]piperidin-1-yl}sulphonyl)-1-phenylethyl]formamide;
(R/S)-2-{[4-(2-fluoro-4-nitrophenoxy)piperidin-1-yl]sulphonyl}-1-phenylethyl(hydroxy)formamide;
(R/S)-2-{[4-(2-chloro-4-methylphenoxy)piperidin-1-yl]sulphonyl}-1-phenylethyl(hydroxy)formamide;
(R/S)-2-{[4-(2-chloro-4-methoxyphenoxy)piperidin-1-yl]sulphonyl}-1-phenylethyl(hydroxy)formamide;
(R/S)-2-{[4-(4-fluoro-2-methoxyphenoxy)piperidin-1-yl]sulphonyl}-1-phenylethyl(hydroxy)formamide;
(R/S)-2-{[4-(4-chloro-2-fluorophenoxy)piperidin-1-yl]sulphonyl}-1-phenylethyl(hydroxy)formamide;
(R/S)-2-{[4-(4-fluoro-2-methylphenoxy)piperidin-1-yl]sulphonyl}-1-phenylethyl(hydroxy)formamide;
(R/S)-2-({4-[(3-chloropyridin-2-yl)oxy]piperidin-1-yl}sulphonyl)-1-pyridin-3-ylethyl(hydroxy)formamide;
(R/S)-2-({4-[(3-cyanopyridin-2-yl)oxy]piperidin-1-yl}sulphonyl)-1-pyridin-3-ylethyl(hydroxy)formamide;
(R/S)-2-({4-[(8-chloroquinolin-4-yl)oxy]-piperidin-1-yl}sulphonyl)-1-pyridin-3-ylethyl(hydroxy)formamide;
(R/S)-hydroxy{1-pyridin-3-yl-2-[(4-{[3-(trifluoromethyl)pyridin-2-yl]oxy}piperidin-1-yl)sulphonyl]ethyl}formamide;
(R/S)-2-[(4-{[3-chloro-5-(trifluoromethyl)pyridin-2-yl]oxy}piperidin-1-yl)sulphonyl]-1-pyridin-3-ylethyl(hydroxy)formamide;
(R/S)-2-[(4-{[3-chloro-5-chloropyridin-2-yl]oxy}piperidin-1-yl)sulphonyl]-1-pyridin-3-ylethyl(hydroxy)formamide;
(R/S)-hydroxy[2-({4-(5-methylthieno[2,3-d]pyrimidin-4-yl)oxy]piperidin-1-yl}sulphonyl)-1-pyridin-3-ylethyl]formamide;
(R/S)-hydroxy[2-({4-[(7-methylthieno[3,2-d]pyrimidin-4-yl)oxy]piperidin-1-yl}sulphonyl)-1-pyridin-3-ylethyl]formamide;
(R/S)-2-({[4-(2-bromo-4-fluorophenoxy)piperidin-1-yl)sulphonyl}methyl)-N-hydroxy-4-methylpentanamide;
(R/S)-3-{[4-(2-bromo-4-fluorophenoxy)piperidin-1-yl]sulphonyl}-2-cyclopentyl-N-hydroxypropanamide;
(R/S)-1-[({4-[1-napthyloxy]piperidin-1-yl}sulphonyl)methyl]-4-pyrimidin-2-ylbutyl(hydroxy)formamide;
(R/S)-1-[({4-[2-chloro-4-fluorophenoxy]piperidin-1-yl}sulphonyl)methyl]-4-pyrimidin-2-ylbutyl(hydroxy)formamide;
(R/S)-1-[({4-[2-bromo-4,6-difluorophenoxy]piperidin-1-yl}sulphonyl)methyl]-4-pyrimidin-2-ylbutyl(hydroxy)formamide;
(R/S)-1-[({4-[2,4-dichlorophenoxy]piperidin-1-yl}sulphonyl)methyl]-4-pyrimidin-2-ylbutyl(hydroxy)formamide; and
(R/S)-1-[({4-[2-cyanophenoxy]piperidin-1-yl}sulphonyl)methyl]-4-pyrimidin-2-ylbutyl(hydroxy)formamide.

Further compounds of the invention, which may be listed with the compounds named above are any one of:
(R/S)-2-{[4-(2-isoxazol-5-ylphenoxy)piperidin-1-yl]sulphonyl}-1-phenylethyl(hydroxy)formamide
(R/S)-hydroxy(1-phenyl-2-{[4-(2-piperidin-1-ylphenoxy)piperidin-1-yl]sulphonyl}ethyl)formamide;
(R/S)-2-({[4-fluoro-2-thien-3-ylphenoxy)piperidin-1-yl]sulphonyl}methyl)-N-hydroxy-4-methylpentanamide; and
(R/S)-2-({[4-(fluoro-2-pyridin-3-ylphenoxy)piperidin-1-yl]sulphonyl}methyl)-N-hydroxy-4-methylpentanamide.

In another aspect the present invention provides a process for the preparation of a compound of formula (1) or a pharmaceutically acceptable salt or in vivo hydrolysable ester thereof wherein Z is —N(OH)CHO, which process comprises the steps of:
a) converting a hydroxylamine of formula (2) into a compound of formula (1); 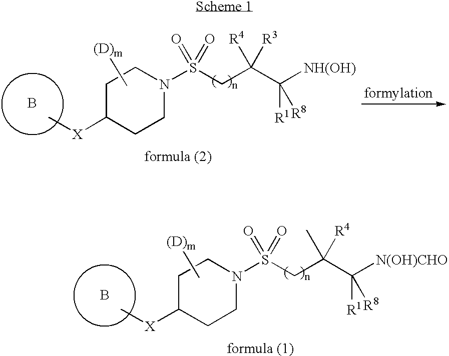
and thereafter if necessary:
i) converting a compound of formula (1) into another compound of formula (1);
ii) removing any protecting groups;
iii) forming a pharmaceutically acceptable salt or in vivo hydrolysable ester.
Formylation may be suitably performed by adding a preformed mixture of acetic acid (8 equivalents) and formic acid (excess) to formula (2) in tetrahydrofuran or dichloromethane so and stirring the solution for 15 hours at temperatures ranging from 0° C. to room temperature followed by stirring in methanol. Alternatively a formylation method described in J. Med. Chem., 2002, 45, 219 using trifluoroethylformate can be used.

This process may further comprise a process for the preparation of a hydroxylamine of formula (2):
when n is 0 and R⁴ is hydrogen (indicated as a compound of formula (2′)), which process comprises:
   b) converting an alkene of formula (3) into a hydroxylamine of formula (2′); 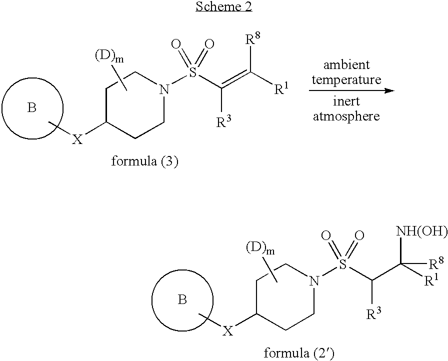
   Suitable reagents for such a conversion include aqueous hydroxylamine in tetrahydrofuran under an argon atmosphere.

The alkene of formula (3) where R⁸ is hydrogen can be prepared by the reaction of a compound of formula (4′) with a compound of formula (5) under Wadsworth-Emmons or Peterson reaction conditions; 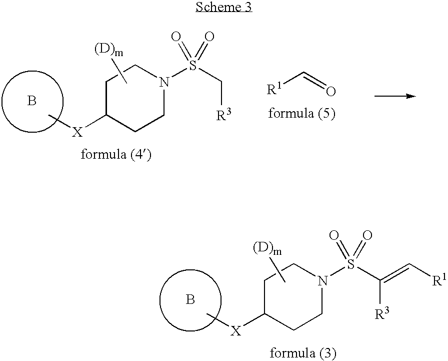
Wadsworth-Emmons or Peterson reactions involve the forming of the anion of formula (4′) with 2 equivalents of lithium bis(trimethylsilyl)amide or sodium hydride or lithium diisopropylamide in tetrahydrofuran at temperatures of −78° C. to 0° C. and reacting this with 1 equivalent of diethylchlorophosphate (Wadsworth Emmons) or 1 equivalent of trimethylsilyl chloride (Peterson). After 1 hour an aldehyde (1.1 equivalent) in tetrahydrofuran is added to the resultant anion described and reacted at room temperature over 15 h.

The alkene of formula (3) can also be prepared by the reaction of a compound of formula (4′) with a compound of formula (6) as illustrated by scheme 4; 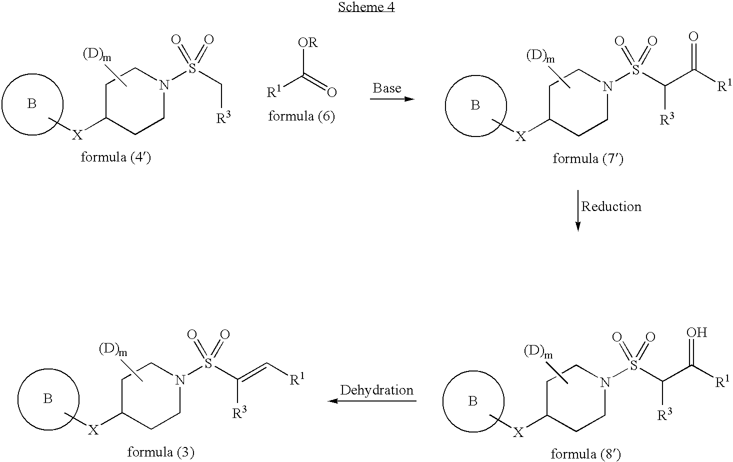
Suitable bases include lithium bis(trimethylsilyl)amide, sodium hydride or lithium diisopropyl amide in tetrahydrofuran at temperatures of −78° C. to 0° C. to form the anion. Suitable reducing agents for the reduction step include sodium borohydride in ethanol or borane-dimethylsulphide complex or borane-tetrahydrofuran complex in tetrahydrofuran at room temperature. Suitable dehydration reagents for the dehydration step include methanesulphonyl chloride or tosyl chloride and triethylamine in dichloromethane at room temperature.

Or a process for the preparation of a hydroxylamine of formula (2):

when n is 0 (indicated as a compound of formula (2^{#})) may comprise;

c)

i) reacting a compound of formula (4″) (see scheme 13 for its preparation) with R¹COOR, R¹COCl or activated R¹COOR to yield a ketone of formula (7″) (where R is C₁₋₂₀alkyl e.g. methyl, ethyl or arylC₁₋₄alkyl e.g. benzyl);
ii) reducing the ketone of formula (7″) to yield an alcohol of formula (8″);
iii) converting —OH group of the alcohol of formula (8″) into a leaving group (L) such as a halide, mesylate, tosylate etc. (see compound of formula (9″);
iv) displacing the leaving group with aqueous hydroxylamine to yield a hydroxylamine of formula (2^{#}); 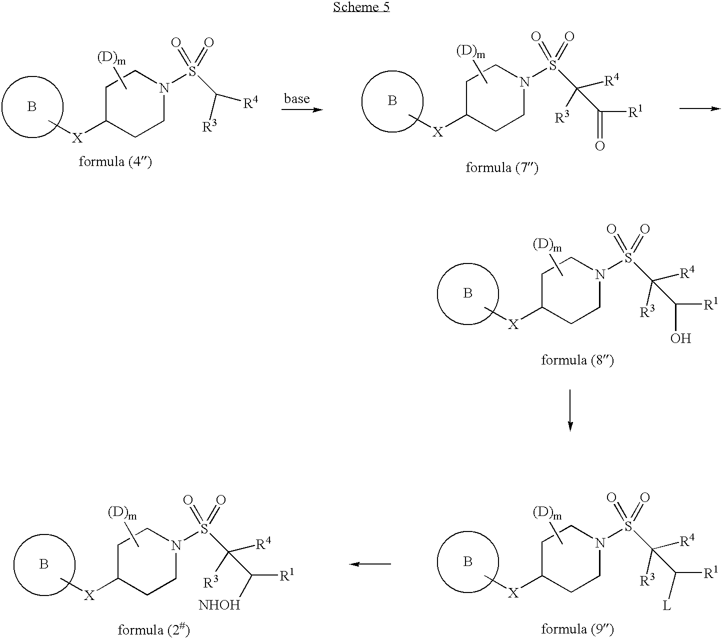
   A ketone of formula (7″) may additionally be prepared by the process illustrated in scheme 6: 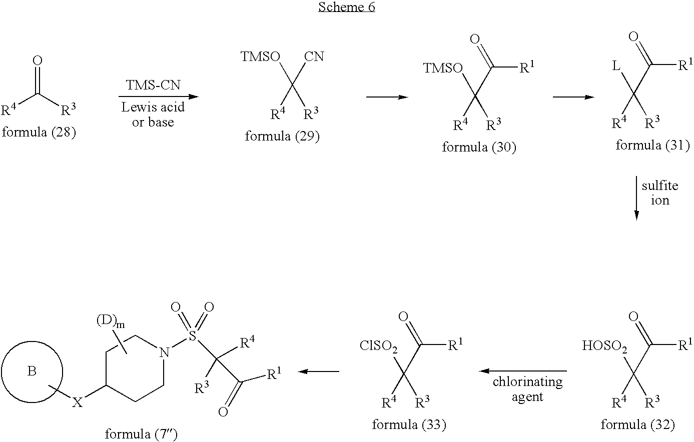
   The silyl group present in the compound of formula (30) can be removed by tetrabutylammonium fluoride. Suitable leaving groups (L) are halo, mesyl and tosyl. A suitable chlorinating agent is POCl₃. A compound of formula (7″) is prepared in the last stage by reacting the compound of formula (33) with the appropriate piperidine reagent.

Or a process for the preparation of a hydroxylamine of formula (2):

when n is 1 and R³ and R⁴ are both hydrogen (indicated as a compound of formula (2**)) may further comprise:

d)

i) reacting a compound of formula (4″) with a compound of formula (10) (either an epoxide or equivalent) to yield an alcohol of formula (8**);
ii) converting —OH group of the alcohol of formula (8**) into a leaving group such as a halide, mesylate, tosylate etc. (see compound of formula (9**);
iii) displacing the leaving group with aqueous hydroxylamine to yield a hydroxylamine of formula (2**); 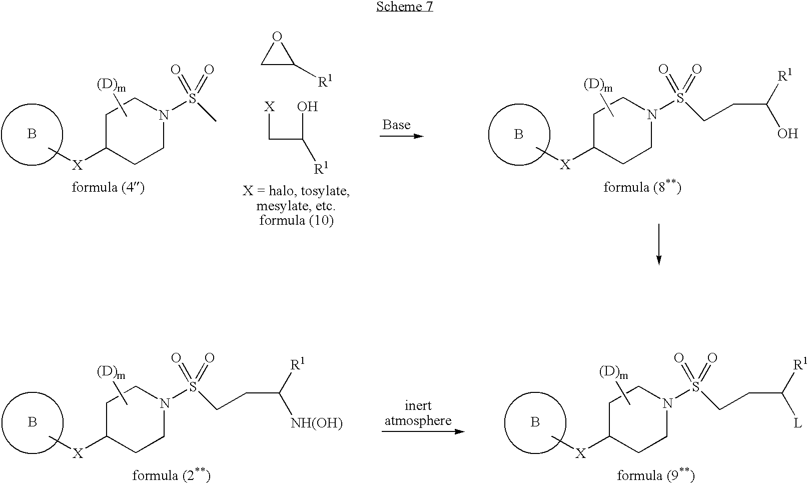
   Suitable bases are lithium bis(trimethylsilyl)amide and lithium diisopropylamide at temperatures from −78° C. to 0° C. Suitable leaving groups (L) are chloro, bromo, iodo, methanesulphonyl and tosyl and these would be formed from the alcohol by treatment with methanesulphonyl chloride and pyridine in dichloromethane (mesylate), tosyl chloride and pyridine in dichloromethane (tosylate), triphenylphosphine and carbon tetrabromide (bromo); the chloro, bromo and iodo derivatives could also be prepared from the mesylate or tosylate by addition of a suitable halide source, e.g. tetrabutylammonium iodide or sodium iodide or lithium chloride in a solvent such as acetone.

Or a process for the preparation of a hydroxylamine of formula (2):

when n is 1, indicated as a compound of formula (2ˆ), may further comprise:

e)

i) reacting a compound of formula (4″) with a compound of formula (11) to yield an ester of formula (12ˆ);
ii) converting the ester of formula (12ˆ) into an alcohol of formula (13ˆ);
iii) displacing the —OH group with aqueous hydroxylamine to yield a hydroxylamine of formula (2ˆ);

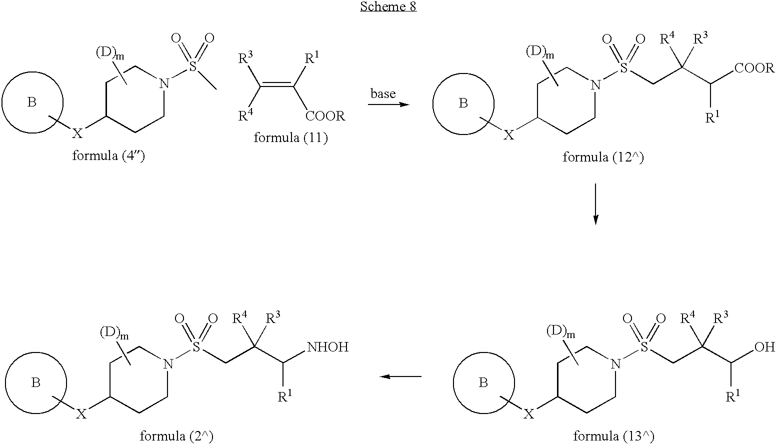

The group —COOR of formula (12ˆ) is representative of an ester wherein R may be C₁₋₂₀ alkyl, e.g. methyl, ethyl or arylC₁₋₄alkyl, e.g. benzyl. Baeyer-Villiger reaction conditions such as a peracid e.g. m-CPBA (3-chloroperoxybenzoic acid) in dichloromethane are suitable for the conversion of the ester group into the alcohol group. It may be appropriate to convert the alcohol group into a leaving group such as bromo, iodo, mesyl and tosyl, before displacement with aqueous hydroxylamine.

In another aspect the present invention provides a process for the preparation of a compound of formula (1) or a pharmaceutically acceptable salt or in vivo hydrolysable ester thereof wherein Z is —CONR¹⁵OH, which process comprises:
a) converting an acid of formula (14) into a compound of formula (1); 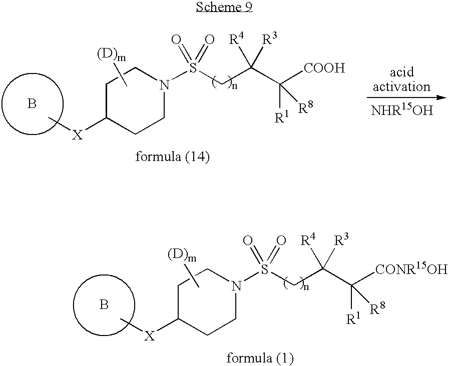
and thereafter if necessary:
i) converting a compound of formula (1) into another compound of formula (1);
ii) removing any protecting groups;
iii) forming a pharmaceutically acceptable salt or in vivo hydrolysable ester.
The acid of formula (14) may be suitably activated by conversion to an acid halide, such as the acid chloride or to an activated ester using carbonyldiimidazole, a carbodiimide or a pentafluorophenyl ester. Alternatively, when the acid of formula (14) is an ester e.g. the methyl or ethyl ester, it can be converted directly to a compound of formula (1) by reaction with NHR¹⁵OH.

Also provided is a process for the preparation of an acid of formula (14) which process comprises;
b) reacting a compound of formula (4″) with an alkene of formula (11) to yield an ester of formula (12ˆ) which is hydrolysed to an acid of formula (14′) where an acid of formula (14′) is an acid of formula (14) wherein n is 1 and R⁸ is hydrogen; 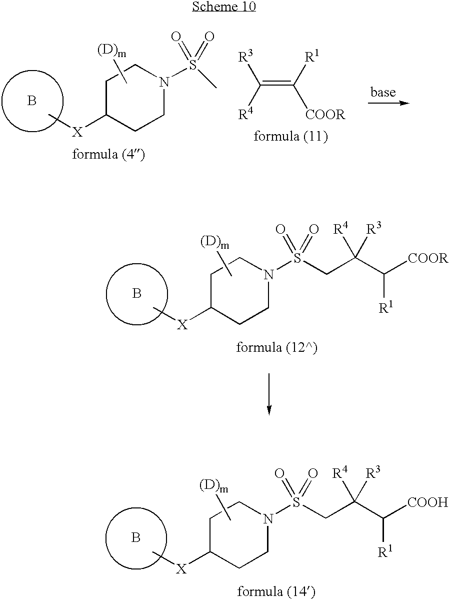
Suitable bases able to deprotonate a compound of formula (4″) include butyllithium, lithium diisopropylamide and lithium bis(trimethylsilyl)amide followed by the addition of a copper salt e.g. copper bromide-dimethylsulphide complex, copper iodide, in solvents such as dimethylsulphide, ether or tetrahydrofuran at temperatures from −78° C. to room temperature.

Or a process for the preparation of an acid of formula (14) comprises;
c) reacting a compound of formula (4″) with a compound of formula (15) to yield an acid of formula (14**) which is an acid of formula (14) wherein n is 0, R³ is hydrogen and R⁴ is hydrogen; 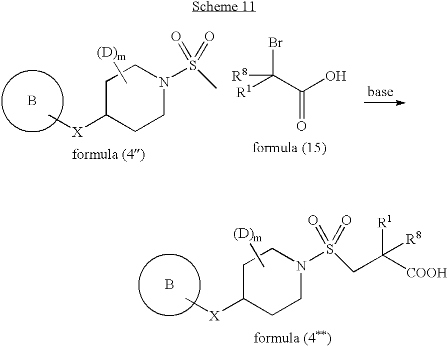
Suitable bases to deprotonate formula (4″) include lithium diisopropylamide, lithium bis(trimethylsilyl)amide and sodium hydride in solvents such as tetrahydrofuran or ether at temperatures from −78° C. to 0° C.

In another aspect the present invention provides a process for the preparation of a compound of formula (1) or a pharmaceutically acceptable salt or in vivo hydrolysable ester thereof wherein Z is —CONR¹⁵OH, R⁸ is hydrogen and n is 0, which process comprises steps as outlined in scheme 12: 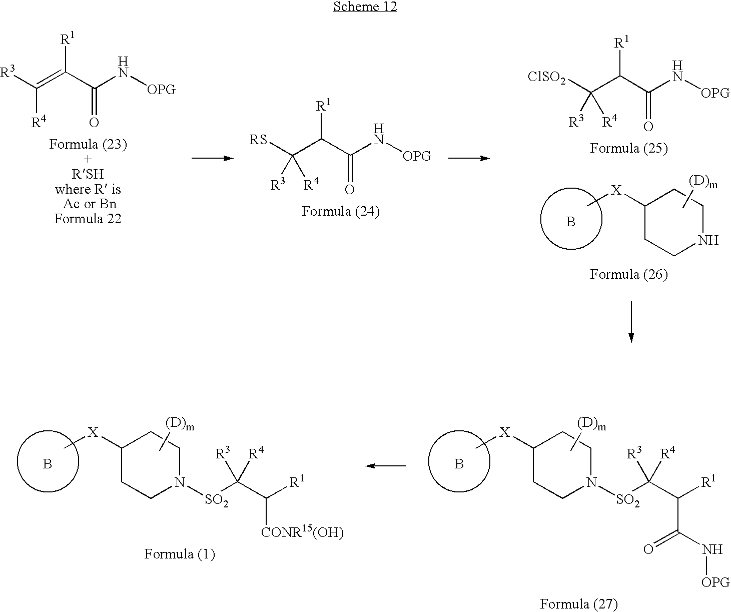
The process of scheme 12 comprises the steps of:
i) reacting a thiol of formula (22) with an acrylate of formula (23) at temperatures of 0° C. to 70° C. to yield a thioether of formula (24);
ii) oxidising the thioether of formula (24) to a sulphonyl chloride of formula (25) by bubbling chlorine gas onto a solution of the thioether in acetic acid at temperatures of 0° C. to room temperature;
iii) reacting the sulphonyl chloride of formula (25) with a piperidine of formula (26) under standard sulphonamide conditions (e.g. triethylamine in DCM at temperatures from 0° C. to 50° C.) to yield a compound of formula (27);
iv) removing the protecting group to yield a compound of formula (1).
   The protecting group (PG) may be benzyl or 2,4-dimethoxybenzyl. The former can be removed by treatment with hydrogen/palladium and the latter by treatment with mild acid (see Tetrahedron Letters, 1998, 39(43), 7865). The process of scheme 12 may further comprise if necessary:
v) converting a compound of formula (1) into another compound of formula (1);
vi) removing any other protecting groups;
vii) forming a pharmaceutically acceptable salt or in vivo hydrolysable ester.

In another aspect of the invention, there is provided a process for the preparation of compounds of formula (4), formula (4′) and formula (4″) which process comprises;
i) reacting a compound of formula (16) where B is an activate halo heterocyclyl with a compound of formula (17) (wherein X is O or S), in the presence of a base to deprotonate the compound of formula (17), to yield a compound of formula (18);
ii) removing the protecting group (PG) from the compound of formula (18) to yield a compound of formula (19);.
iii) reacting the compound of formula (19) with a suitable reagent to yield a compound of formula (4); and
iv) oxidising X where X is S as required.
When R⁴ is hydrogen a compound of formula (4′) is produced and when R³ and R⁴ are both hydrogen compound of formula (4″) is produced; 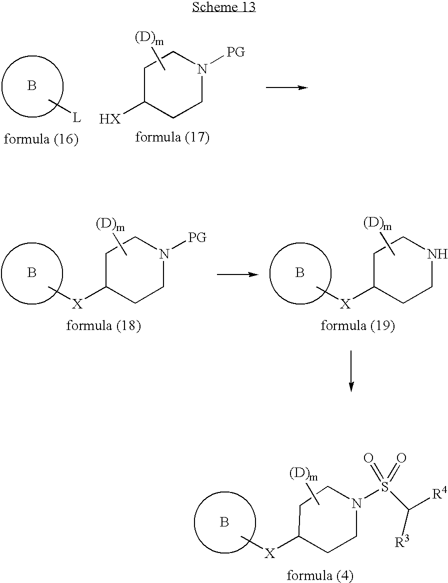
   Compounds of formula (4), formula (4′) and formula (4″) may also be prepared by a process which comprises;
i) reacting a compound of formula (20) (wherein X is O or S) with a compound of formula (21), in the presences of a base to yield a compound of formula (18);
ii) removing the protecting group (PG) from the compound of formula (18) to yield a compound of formula (19);.
iii) reacting the compound of formula (19) with a suitable reagent to yield a compound of formula (4); and
iv) oxidising X as required.
When R⁴ is hydrogen a compound of formula (4′) is produced and when R³ and R⁴ are both hydrogen compound of formula (4″) is produced; 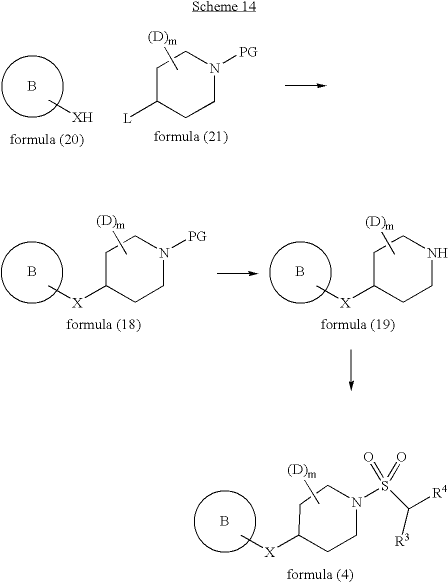
   In both schemes 13 and 14: L is a suitable leaving group such as halo (chloro, bromo, iodo), hydroxy, mesyl and tosyl; suitable bases to deprotonate compounds of formula (17) and formula (20) include sodium hydride, lithium diisopropylamide, lithium bis(trimethylsilyl) de and butyllithium; suitable reaction conditions for a) are temperatures ranging from −78° C. to 70° C. and an aprotic solvent, e.g. tetrahydrofuran under argon; suitable protecting groups (PG) include Boc (t-butoxycarbonyl), CBz (carbonyloxybenzyl) groups and mesyl or another alkylsulphonyl. In the case where PG is alkylsulphonyl, reaction of formula (16) and (17) and of formula (20) and formula (21) directly produces a compound of formula (4). A compound of formula (18) can be converted to formula (19) by treatment with acid (Boc) or hydrogen/palladium (CBz). A compound of formula (19) can be converted to a compound of formula (4) by treatment with an alkylsuphonyl chloride in the presence of a base such as pyridine in a solvent such as dichloromethane. When B is aromatic, X is O and L is OH, Mitsunobu conditions can be used to form a compound of formula (18), i.e. a compound of formula (16) or formula (20) would be reacted with a mixture of diethyl azodicaboxylate or diisopropylazodicarboxylate and triphenylphosphine and formula (17) or formula (21) to give a compound of formula (4). In addition PG could also be a protected hydroxamic acid or reverse hydroxamate. Thus reaction of formula (16) and (17) and of formula (20) and (21) would deliver a protected version of formula (1) which could then be deprotected.

A compound of formula (1) can be prepared by removal of a protecting group on the zinc binding group directly. The protecting group (PG) can be benzyl or 2,4-dimethoxybenzyl. The former can be removed by treatment with hydrogen/palladium and the latter by treatment with mild acid (see Tetrahedron Letters, 1998, 39(43), 7865). The required protected hydroxamic acid or reverse hydroxamate can be obtained by using a suitably protected hydroxylamine earlier in the synthesis.

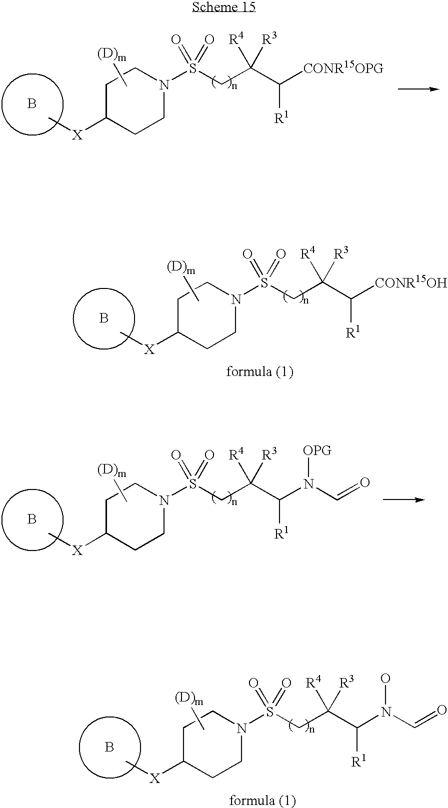

It will be appreciated that certain of the various zinc substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogen group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or tert-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a tert-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

As stated hereinbefore the compounds defined in the present invention possesses metalloproteinases inhibitory activity, and in particular TACE inhibitory activity. This property may be assessed, for example, using the procedure set out below.

Isolated Enzyme Assays

Matrix Metalloproteinase Family Including for Example MMP13.

Recombinant human proMMP13 may be expressed and purified as described by Knauper et al. [V. Knauper et al., (1996) The Biochemical Journal 271:1544-1550 (1996)]. The purified enzyme may be used to monitor inhibitors of activity as follows: purified proMMP13 is activated using 1 mM amino phenyl mercuric acid (APMA), 20 hours at 21° C.; the activated MMP13 (11.25 ng per assay) is incubated for 4-5 hours at 35° C. in assay buffer (0.1M Tris-HCl, pH 7.5 containing 0.1M NaCl, 20 mM CaCl2, 0.02 mM ZnCl and 0.05% (w/v) Brij 35 using the synthetic substrate 7-methoxycoumarin-4 yl)acetyl.Pro.Leu.Gly.Leu.N-3-(2,4-dinitrophenyl)-L-2,3-diaminopropionyl.Ala.Arg.NH₂ in the presence or absence of inhibitors. Activity is determined by measuring the fluorescence at λex 328 nm and λem 393 nm. Percent inhibition may be calculated as follows: % Inhibition is equal to the [Fluorescence_{plus inhibitor}−Fluorescence_{background}] divided by the [Fluorescence_{minus inhibitor}−Fluorescence_{background}].

A similar protocol can be used for other expressed and purified pro MMPs using substrates and buffers conditions optimal for the particular MMP, for instance as described in C. Graham Knight et al., (1992) FEBS Lett. 296(3):263-266.

Adamalysin Family Including for Example TNF Convertase

The ability of the compounds to inhibit proTNFα convertase enzyme (TACE) has been assessed using a partially purified, isolated enzyme assay, the enzyme being obtained from the membranes of THP-1 as described by K. M. Mohler et al., (1994) Nature 370:218-220. The purified enzyme activity and inhibition thereof was determined by incubating the partially purified enzyme in the presence or absence of test compounds using the substrate 4′,5′-Dimethoxy-fluoresceinyl Ser.Pro.Leu.Ala.Gln.Ala.Val.Arg.Ser.Ser.Ser.Arg.Cys(4-(3-succinimid-1-yl)fluorescein)-NH₂ in assay buffer (50 mM Tris HCl, pH 7.4 containing 0.1% (w/v) Triton X-100 and 2 mM CaCl₂), at 26° C. for 4 hours. The amount of inhibition was determined as for MMP13 except λex 485 nm and λem 538 nm were used. The substrate was synthesised as follows. The peptidic part of the substrate was assembled on Fmoc-NH-Rink-MBHA-polystyrene resin either manually or on an automated peptide synthesiser by standard methods involving the use of Fmoc-amino acids and O-benzotriazol-1-yl-N,N,N′,N′-tetramethyluronium hexafluorophosphate (HBTU) as coupling agent with at least a 4- or 5-fold excess of Fmoc-amino acid and HBTU. Ser¹ and Pro² were double-coupled. The following side chain protection strategy was employed; Ser¹(But), Gln⁵(Trityl), Arg^{8,12}(Pmc or Pbf), Ser^{9,10,11}(Trityl), Cys¹³(Trityl). Following assembly, the N-terminal Fmoc-protecting group was removed by treating the Fmoc-peptidyl-resin with in DMF. The amino-peptidyl-resin so obtained was acylated by treatment for 1.5-2 hr at 70° C. with 1.5-2 equivalents of 4′,5′-dimethoxy-fluorescein-4(5)-carboxylic acid [Khanna & Ullman, (1980) Anal Biochem. 108:156-161) which had been preactivated with diisopropylcarbodiimide and 1-hydroxybenzotriazole in DMF]. The dimethoxyfluoresceinyl-peptide was then simultaneously deprotected and cleaved from the resin by treatment with trifluoroacetic acid containing 5% each of water and triethylsilane. The dimethoxyfluoresceinyl-peptide was isolated by evaporation, trituration with diethyl ether and filtration. The isolated peptide was reacted with 4-(N-maleimido)-fluorescein in DMF containing diisopropylethylamine, the product purified by RP-HPLC and finally isolated by freeze-drying from aqueous acetic acid. The product was characterised by MALDI-TOF MS and amino acid analysis.

The compounds of the invention have been found to be active against TACE at 0.1 nM to 50 μM and in particular 10 μM of compound 8 gave 81% inhibition and 10 μM of compound 14 gave 76% inhibition.

Natural Substrates

The activity of the compounds of the invention as inhibitors of aggrecan degradation may be assayed using methods for example based on the disclosures of E. C. Arner et al., (1998) Osteoarthritis and Cartilage 6:214-228; (1999) Journal of Biological Chemistry, 274 (10), 6594-6601 and the antibodies described therein. The potency of compounds to act as inhibitors against collagenases can be determined as described by T. Cawston and A. Barrett (1979) Anal. Biochem. 99:340-345.

Inhibition of Metalloproteinase Activity in Cell/Tissue Based Activity

Test as an Agent to Inhibit Membrane Sheddases such as TNF Convertase

The ability of the compounds of this invention to inhibit the cellular processing of TNFα production may be assessed in THP-1 cells using an MESA to detect released TNF essentially as described K. M. Mohler et al., (1994) Nature 370:218-220. In a similar fashion the processing or shedding of other membrane molecules such as those described in N. M. Hooper et al., (1997) Biochem. J. 321:265-279 may be tested using appropriate cell lines and with suitable antibodies to detect the shed protein.

Test as an Agent to Inhibit Cell Based Invasion

The ability of the compound of this invention to inhibit the migration of cells in an invasion assay may be determined as described in A. Albini et al., (1987) Cancer Research 47:3239-3245.

Test as an Agent to Inhibit Whole-Blood TNF Sheddase Activity

The ability of the compounds of this invention to inhibit TNFα production is assessed in a human whole blood assay where LPS is used to stimulate the release of TNFα. 160 μl of heparinized (10 Units/ml) human blood obtained from volunteers, was added to the plate and incubated with 20 μl of test compound (duplicates), in RPMI1640+bicarbonate, penicillin, streptomycin, glutamine and 1% DMSO, for 30 minutes at 37° C. in a humidified (5% CO₂/95% air) incubator, prior to addition of 20 μl LPS (E. coli. 0111:B4; final concentration 10 μg/ml). Each assay includes controls of neat blood incubated with medium alone or LPS (6 wells/plate of each). The plates are then incubated for 6 hours at 37° C. (humidified incubator), centrifuged (2000 rpm for 10 min; 4° C.), plasma harvested (50-100 μl) and stored in 96 well plates at −70° C. before subsequent analysis for TNFα concentration by ELISA.

Test as an Agent to Inhibit In Vitro Cartilage Degradation

The ability of the compounds of this invention to inhibit the degradation of the aggrecan or collagen components of cartilage can be assessed essentially as described by K. M. Bottomley et al., (1997) Biochem J. 323:483-488.

In Vivo Assessment

Test as an Anti-TNF Agent

The ability of the compounds of this invention as in vivo TNFα inhibitors is assessed in the rat. Briefly, groups of female Wistar Alderley Park (AP) rats (90-100 g) are dosed with compound (5 rats) or drug vehicle (5 rats) by the appropriate route e.g. peroral (p.o.), intraperitoneal (i.p.), subcutaneous (s.c.) 1 hour prior to lipopolysaccharide (LPS) challenge (30 μg/rat i.v.). Sixty minutes following LPS challenge rats are anaesthetised and a terminal blood sample taken via the posterior vena cavae. Blood is allowed to clot at room temperature for 2 hours and serum samples obtained. These are stored at −20° C. for TNFα ELISA and compound concentration analysis.

Data analysis by dedicated software calculates for each compound/dose:

$$Percent inhibition of TNF α = \frac{\begin{matrix}Mean TNF α ( Vehicle control ) - \\ Mean TNF α ( Treated ) \times 100\end{matrix}}{Mean TNF α ( Vehicle control )}$$

Test as an Anti-Arthritic Agent

Activity of a compound as an anti-arthritic is tested in the collagen-induced arthritis (CIA) as defined by D. E. Trentham et al., (1977) J. Exp. Med. 146,:857. In this model acid soluble native type II collagen causes polyarthritis in rats when administered in Freunds incomplete adjuvant. Similar conditions can be used to induce arthritis in mice and primates.

Pharmaceutical Compositions

According to a further aspect of the invention there is provided a pharmaceutical composition which comprises a compound of formula (1), or a pharmaceutically acceptable salt or in vivo hydrolysable ester thereof, as defined hereinbefore in association with a pharmaceutically-acceptable diluent or carrier.

The composition may be in a form suitable for oral administration, for example as a tablet or capsule, for parenteral injection (including intravenous, subcutaneous, intramuscular, intravascular or infusion) as a sterile solution, suspension or emulsion, for topical administration as an ointment or cream or for rectal administration as a suppository. The composition may also be a form suitable for inhalation

In general the above compositions may be prepared in a conventional manner using conventional excipients.

The pharmaceutical compositions of this invention will normally be administered to humans so that, for example, a daily dose of 0.5 to 75 mg/kg body weight (and preferably 0.5 to 30 mg/kg body weight) is received. This daily dose may be given in divided doses as necessary, the precise amount of the compound received and the route of administration depending on the weight, age and sex of the patient being treated and on the particular disease condition being treated according to principles known in the art.

Typically unit dosage forms will contain about 1 mg to 500 mg of a compound of this invention.

Therefore in a further aspect of the present invention there is provided a compound of formula (1), or a pharmaceutically acceptable salt or in vivo hydrolysable ester thereof, as defined hereinbefore, for use in a method of treatment of a warm-blooded animal such as man by therapy.

Also provided is a compound of formula (1), or a pharmaceutically acceptable salt or in vivo hydrolysable ester thereof, as defined hereinbefore, for use in a method of treating a disease condition mediated by one or more metalloproteinase enzymes and in particular a disease condition mediated by TNFα.

Further provided is a compound of formula (1), or a pharmaceutically acceptable salt or in vivo hydrolysable ester thereof, as defined hereinbefore, for use in a method of treating inflammatory diseases, autoimmune diseases, allergic/atopic diseases, transplant rejection, graft versus host disease, cardiovascular disease, reperfusion injury and malignancy in a warm-blooded animal such as man. In particular a compound of formula (1), or a pharmaceutically acceptable salt or in vivo hydrolysable ester thereof, as defined hereinbefore, is provided for use in a method of treating rheumatoid arthritis, Crohn's disease and psoriasis, and especially rheumatoid arthritis. A compound of formula (1), or a pharmaceutically acceptable salt or in vivo hydrolysable ester thereof, as defined hereinbefore, is also provided for use in a method of treating a respiratory disorder such as asthma or COPD.

According to an additional aspect of the invention there is provided a compound of formula (1), or a pharmaceutically acceptable salt or in vivo hydrolysable ester thereof, as defined hereinbefore, for use as a medicament.

Also provided is a compound of formula (1), or a pharmaceutically acceptable salt or in vivo hydrolysable ester thereof, as defined hereinbefore, for use as a medicament in the treatment of a disease condition mediated by one or more metalloproteinase enzymes and in particular a disease condition mediated by TNFα.

Further provided is a compound of formula (1), or a pharmaceutically acceptable salt or in vivo hydrolysable ester thereof, as defined hereinbefore, for use as a medicament in the treatment of inflammatory diseases, autoimmune diseases, allergic/atopic diseases, transplant rejection, graft versus host disease, cardiovascular disease, reperfusion injury and malignancy in a warm-blooded animal such as man. In particular a compound of formula (1), or a pharmaceutically acceptable salt or in vivo hydrolysable ester thereof, as defined hereinbefore, is provided for use as a medicament in the treatment of rheumatoid arthritis, Crohn's disease and psoriasis, and especially rheumatoid arthritis. A compound of formula (1), or a pharmaceutically acceptable salt or in vivo hydrolysable ester thereof, as defined hereinbefore, is also provided for use as a medicament in the treatment of a respiratory disorder such as asthma or COPD.

According to this another aspect of the invention there is provided the use of a compound of formula (1), or a pharmaceutically acceptable salt or in vivo hydrolysable ester thereof, as defined hereinbefore in the manufacture of a medicament for use in the treatment of a disease condition mediated by one or more metalloproteinase enzymes and in particular a disease condition mediated by TNFα in a warm-blooded animal such as man.

Also provided is the use of a compound of formula (1), or a pharmaceutically acceptable salt or in vivo hydrolysable ester thereof, as defined hereinbefore in the manufacture of a medicament for use in the treatment of inflammatory diseases, autoimmune diseases, allergic/atopic diseases, transplant rejection, graft versus host disease, cardiovascular disease, reperfusion injury and malignancy in a warm-blooded animal such as man. In particular the use of a compound of formula (1), or a pharmaceutically acceptable salt or in vivo hydrolysable ester thereof, as defined hereinbefore, is provided in the manufacture of a medicament in the treatment of rheumatoid arthritis, Crohn's disease and psoriasis, and especially rheumatoid arthritis. The use of a compound of formula (1), or a pharmaceutically acceptable salt or in vivo hydrolysable ester thereof, as defined hereinbefore, is provided in the manufacture of a medicament in the treatment of a respiratory disorder such as asthma or COPD.

According to a further feature of this aspect of the invention there is provided a method of producing a metalloproteinase inhibitory effect in a warm-blooded animal, such as man, in need of such treatment which comprises administering to said animal an effective amount of a compound of formula (1).

According to a further feature of this aspect of the invention there is provided a method of producing a TACE inhibitory effect in a warm-blooded animal, such as man, in need of such treatment which comprises administering to said animal an effective amount of a compound of formula (1).

According to this further feature of this aspect of the invention there is provided a method of treating autoimmune disease, allergic/atopic diseases, transplant rejection, graft versus host disease, cardiovascular disease, reperfusion injury and malignancy in a warm-blooded a such as man, in need of such treatment which comprises administering to said animal an effective amount of a compound of formula (1).

Also provided is a method of treating rheumatoid arthritis, Crohn's disease and psoriasis, and especially rheumatoid arthritis in a warm-blooded animal, such as man, in need of such treatment which comprises administering to said animal an effective amount of a compound of formula (1). Further provided is a method of treating a respiratory disorder such as asthma or COPD in a warm-blooded animal, such as man, in need of such treatment which comprises administering to said animal an effective amount of a compound of formula (1).

In addition to their use in therapeutic medicine, the compounds of formula (1) and their pharmaceutically acceptable salts are also useful as pharmacological tools in the development and standardisation of in vitro and in vivo test systems for the evaluation of the effects of inhibitors of cell cycle activity in laboratory animals such as cats, dogs, rabbits, monkeys, rats and mice, as part of the search for new therapeutic agents.

In the above other pharmaceutical composition, process, method, use and medicament manufacture features, the alternative and preferred embodiments of the compounds of the invention described herein also apply.

### EXAMPLES

The invention will now be illustrated by the following non-limiting examples in which, unless stated otherwise:

(i) temperatures are given in degrees Celsius (° C.); operations were carried out at room or ambient temperature, that is, at a temperature in the range of 18-25° C.;

(ii) organic solutions were dried over anhydrous magnesium sulphate; evaporation of solvent was carried out using a rotary evaporator under reduced pressure (600-4000 Pascals; 4.5-30 mm Hg) with a bath temperature of up to 60° C.;

(iii) chromatography unless otherwise stated means flash chromatography on silica gel; thin layer chromatography (TLC) was carried out on silica gel plates; where a “Bond Elut” column is referred to, this means a column containing 10 g or 20 g of silica of 40 micron particle size, the silica being contained in a 60 ml disposable syringe and supported by a porous disc, obtained from Varian, Harbor City, Calif., USA under the name “Mega Bond Elut SI”. Where an “Isolute™ SCX column” is referred to, this means a column containing benzenesulphonic acid (non-endcapped) obtained from International Sorbent Technology Ltd., 1st House, Duffryn Industial Estate, Ystrad Mynach, Hengoed, Mid Glamorgan, UK. Where Flashmaster II is referred to, this means a UV driven automated chromatography unit supplied by Jones;

(iv) in general, the course of reactions was followed by TLC and reaction times are given for illustration only;

(v) yields, when given, are for illustration only and are not necessarily those which can be obtained by diligent process development; preparations were repeated if more material was required;

(vi) when given, ¹H NMR data is quoted and is in the form of delta values for major diagnostic protons, given in parts per million (ppm) relative to tetramethylsilane (TMS) as an internal standard, determined at 300 M using perdeuterio DMSO (CD₃SOCD₃) as the solvent unless otherwise stated; coupling constants (I) are given in Hz;

(vii) chemical symbols have their usual meanings; SI units and symbols are used;

(viii) solvent ratios are given in percentage by volume;

(ix) mass spectra (MS) were run with an electron energy of 70 electron volts in the chemical ionisation (APCI) mode using a direct exposure probe; where indicated ionisation was effected by electrospray (ES); where values for m/z are given, generally only ions which indicate the parent mass are reported, and unless otherwise stated the mass ion quoted is the positive mass ion—(M+H)⁺;

(x) LCMS characterisation was performed using a pair of Gilson 306 pumps with Gilson 233 XL sampler and Waters ZMD4000 mass spectrometer. The LC comprised water symmetry 4.6×50 column C18 with 5 micron particle size. The eluents were: A, water with 0.05% formic acid and B, acetonitrile with 0.05% formic acid The eluent gradient went from 95% A to 95% B in 6 minutes. Where indicated ionisation was effected by electrospray (ES); where values for m/z are given, generally only ions which indicate the parent mass are reported, and unless otherwise stated the mass ion quoted is the positive mass ion—(M+H)⁺ and

(xi) the following abbreviations are used:

DMSO dimethyl sulphoxide;

DMF N-dimethylformamide;

DCM dichloromethane;

NMP N-methylpyrrolidinone;

DIAD di-isopropylazodicarboxylate

LHMDS or LiHMDS lithium bis(trimethylsilyl)amide

MeOH methanol

RT room temperature

TFA trifluoroacetic acid

EtOH ethanol

EtOAc ethyl acetate

Et₂O diethylether

THF tetrahydrofuran

TBDMS tertiarybutyldimethylsilyl

DIPEA diisopropylethylamine

MTBE methyltertiarybutylether

The invention will now be illustrated but not limited by the following Examples:

### Example 1

### (R/S)-2-{[4-(2-isoxazol-5-ylphenoxy)piperidin-1-yl]sulphonyl}-1-phenylethyl(hydroxy)formamide

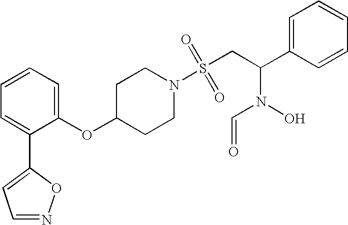

To a solution of (R/S)-2-{[4-(2-isoxazol-5-ylphenoxy)piperidin-1-yl]sulphonyl}-1-phenylethyl(hydroxylamine) (described below) (330 mg, 0.75 mmol) in DCM (0.5 ml) was added a pre-mixture of formic acid (2 ml) and acetic anhydride (1 m) and the reaction stirred at RT overnight. MeOH (5 ml) was then added and the mixture stirred at RT for 1 hour. After evaporation the residues were re-dissolved in MeOH and stirred for 3 hours before re-evaporation. The residue was purified by BondElut chromatography, eluting with a gradient from DCM to 5% methanol in DCM to give (R/S)-2-{[4-(2-isoxazol-5-ylphenoxy)piperidin-1-yl]sulphonyl}-1-phenylethyl(hydroxy)formamide (88 mg, 0.19 mmol). MS: 472.

The starting (R/S)-2-{[4-(2-isoxazol-5-ylphenoxy)piperidin-1-yl]sulphonyl}-1-phenylethyl(hydroxylamine) was prepared as follows:

i. Triethylamine (8.0 g, 0.079 mol) was added to a stirred solution of E-β-styrenesulphonyl chloride (12.0 g, 0.059 mol) and 4-hydroxypiperidine (8.0 g, 0.079 mol) in THF (100 ml) at RT. Stirring was continued overnight before the reaction mixture was reduced to low volume and partitioned between EtOAc followed by aqueous 1M HCl, saturated NaHCO₃ and brine. The organic fraction was then dried (Na₂SO₄) and evaporated to give a solid product. (12.75 g; 0.046 mol); NMR (CDCl₃): 1.5-1.8 (m, 4H), 1.9-2.1 (m, 2H), 3.0-3.2 (m, 2H), 3.4-3.6 (m, 2H), 3.85 (s, 1H), 6.65 (s, 1H), 7.3-7.6 (m, 6H); MS: 268.

ii. 2-(5-Isoxazolyl)-phenol (121 mg, 0.75 mmol) was dissolved in DCM (1 ml) and E-1-(4-hydroxypiperidin-1-ylsulphonyl)-2-phenylethene (0.2 g, 0.75 mmol) was added. A solution of triphenylphosphine (0.2 g, 0.75 mmol) in DCM (2 ml) followed by a solution of DIAD (0.15 ml, 0.75 mmol) in DCM (2 ml) was then added and the resulting mixture, stirred at RT overnight. The mixture was concentrated and purified by chromatography: bond elute cartridge, eluent hexane (5 minutes; 20 ml/minute), 100% hexane to 100% DCM (15 minute) to give E-[4-(2-(5-isoxazolyl)phenyloxy)piperidin-1-ylsulphonyl]-2-phenylethene, which was carried through to the next step.

iii. E-[4-(2-(5-isoxazolyl)phenyloxy)piperidin-1-ylsulphonyl]-2-phenylethene, was dissolved in THF (1 ml) and the air in the tubes excluded with argon before hydroxylamine in water (50% solution, 1 ml) was added and the mixture stirred vigorously overnight. EtOAc (1 ml) was added and the aqueous layer separated. The organic layers were washed with brine and dried (Na₂SO₄) and concentrated to give (R/S)-2-{[4-(2-isoxazol-5-ylphenoxy)piperidin-1-yl]sulphonyl}-1-phenylethyl(hydroxylamine) which was carried through to the final step.

### Example 2

### (R/S)-1-[({4-[2-chloro-4-(trifluoromethyl)phenoxy]piperidin-1-yl}sulphonyl)methyl]-4-pyrimidin-2-ylbutyl(hydroxy)formamide

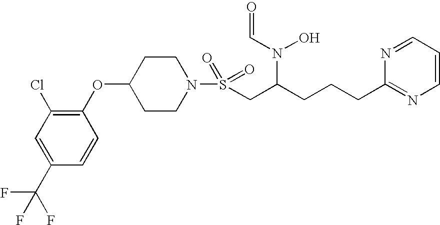

To formic acid (2.32 ml) at 0° C. was added acetic anhydride (0.84 ml). After 20 minutes this was added to (R/S)-1-[({4-[2-chloro-4-(trifluoromethyl)phenoxy]piperidin-1-yl}sulphonyl)methyl]-4-pyrimidin-2-ylbutyl(hydroxylamine) (0.67 g, 1.28 mmol) dissolved in THF (6.9 ml) and formic acid (2.32 ml) and the resulting solution stirred for 10 minutes. The solvent was removed in vacuo and the residue dissolved in DCM, washed with saturated sodium bicarbonate solution, dried and evaporated to dryness. The product was then re-dissolved in MeOH and stirred overnight. The solvent was removed in vacuo and the residue stirred in Et₂O to give (R/S)-1-[({4-[2-chloro-4-(trifluoromethyl)phenoxy]piperidin-1-yl}sulphonyl)methyl]-4-pyrimidin-2-ylbutyl(hydroxy)formamide as a white solid (0.19 g, 0.35 mmol). NMR: (CDCl₃, 300 MHz): 9.99 (s, 0.5H)*; 9.18 (brs, 0.5H)*; 8.70 (dd, 2H); 8.52 (s, 0.5H)*; 8.05 (s, 0.5H)*, 7.67 (s, 1H); 7.48 (d, 1H); 7.21 (t, 1H); 6.99 (d, 1H); 4.91 (m, 0.5H)*, 4.70 (bs,1H); 4.23 (m, 0.5H)*; 3.63-3.27(m, 5H), 3.20-2.85 (m, 2H), 2.10-1.85 (m, 7H, 1.82-1.60(m, 3H); δC (CDCl₃, 75.5 MHz):162.0, 157.6, 157.5, 157.4, 128.3, 125.4, 119.3, 115.0, 72.2, 72.0, 56.1, 51.5, 51.4, 50.5, 42.1, 49.1, 41.7, 37.9, 37.3, 30.5, 30.3, 30.1, 28.6, 24.1, 23.8; MS: 551.42; HPLC: 5%-95% MeOH 10 minutes gradient: 9.088 m, 91.62%.

*rotameric signals

The starting (R/S)-1-[({4-[2-chloro-4-(trifluoromethyl)phenoxy]piperidin-1-yl}sulphonyl)methyl]-4-pyrimidin-2-ylbutyl(hydroxylamine) was prepared as follows:

i. Diisopropyl azodicarboxylate (6.68 ml, 33 mmol) was added dropwise to a solution of tert-butyl 4-hydroxypiperidine carboxylate (4.27 g, 21.2 mmol) and triphenyl phosphine (7.78 g, 29.7 mmol) in toluene (160 ml) at 0° C. under argon. The mixture was stirred for ½ hour, 2-chloro-4-trifluoromethylphenol (5.00 g, 25.5 mmol) was then added dropwise and the reaction allowed to warm to RT overnight. The solvent was removed in vacuo and the residue stirred in isohexane for 1 hour. The precipitate was filtered off and the filtrate concentrated to an orange oil which was purified by flash column chromatography (10% EtOAc in isohexane) to afford 1-tert-butyl-4-(2-chloro-4-trifluoromethylphenyloxy)piperidine carboxylate (4.59 g, 12 mmol). NMR: (CDCl₃, 300 MHz): 7.68 (s, 1H); 7.46 (d, 1); 6.98 (d, 1H); 4.68 (m, 1H); 3.69-3.44 (m, 4H); 1.79-1.92 (m, 4H); 1.46 (s, 9H).

ii. TFA (11.76 ml) was added to a solution of 1-tert-butyl-4-(2-chloro-4-trifluoromethylphenyloxy)piperidine carboxylate (4.59 g, 12 mmol) in DCM (23.5 ml) at 0° C. and the solution stirred for 20 hours. The solvent was removed in vacuo, the residue taken up in 2M aqueous sodium hydroxide solution and water and then extracted into EtOAc. The organics were dried (MgSO₄) and concentrated to give 4-(2-chloro-4-trifluoromethylphenyloxy)piperidine TFA salt as a white solid (4.47 g, 11.4 mmol). NMR (CDCl₃, 300 MHz): 7.66 (s, 1H); 7.50 (d, 1H); 7.00 (d, 1H); 4.83 (bs, 1H); 3.50-3.19 (m, 4H); 2.40-2.11 (m, 4H).

iii. Methanesulphonyl chloride (1.36 ml) was added dropwise to a solution of 4-(2-chloro-4-trifluoromethylphenyloxy)piperidine TFA salt (4.47 g, 11.4 mmol) in triethylamine (6.67 ml) and DCM (58 ml) at 0° C., under argon. The mixture was allowed to come to RT over a weekend. DCM was added to the reaction mixture, the organics were washed with water, dried (MgSO₄) and concentrated in vacuo to give 4-(2-chloro-4-trifluoromethylphenyloxy)piperidin-1-ylsulphonylmethane as an oil (1.43 g, 4 mmol). NMR (CDCl₃, 300 MHz): 7.67 (s, 1H); 7.51 (d, 1H); 7.00 (d, 1H); 4.75 (m, 1H; 359-3.49 (m, 2H); 3.39-3.20(m, 2H); 2.83 (s, 3H); 2.15-2.00 (m, 4H).

iv. LHMDS (6.15 ml of a 1M solution in THF) was added dropwise to a solution of 4-(2-chloro-4-trifluoromethylphenyloxy)piperidin-1-ylsulphonylmethane (1.00 g 2.8 mmol) in THF (11 ml) at −10° C. under argon. The mixture was stirred for 10 minutes and then trimethylsilyl chloride (0.36 ml) was added dropwise at −10° C. Stirring was continued for a further 20 minutes and then 4-(2-pyrimidinyl)butan-1-al§ (462 mg, 3.1 mmol) was added in THF (5 ml) again ensuring that the temperature did not exceed −10° C. The reaction mixture was stirred for 2 hours and then quenched with brine at −10° C. The solution was allowed to warm to RT, diluted with water and the aqueous layer extracted with EtOAc. The organics were dried (MgSO₄) and concentrated to a yellow oil, purified by flash column chromatography (5% MeOH in DCM) to afford E/Z-1-{4-(2-chloro-4-trifluoromethylphenyloxy)piperidin-1-ylsulphonyl}-5-(pyrimidin-2-yl)pent-1-ene (0.64 g, 1.3 mmol). NMR: (CDCl₃, 300 MHz): 8.67 (2×d overlaid, 2H*; 7.64 (m, 1H); 7.46 (bd, 1H); 7.14 (m, 1H); 7.00 (dd, 1H); 6.80 (dt, 0.5H)*; 6.40 (dt, 0.5H)*; 6.18 (d, 0.5H)*; 6.05 (d, 0.5H)*; 4.70 (bs,1H); 3.50-3.32 (m, 2H); 3.29-3.09 (m, 2H); 3.02 (dd, J=7.7 Hz, J=7.7 Hz, 2H, CH₂CH₂Ar); 2.73 (ddd, J=14.9 Hz, J=7.34 Hz, 1H, 1H); 2.39 (ddd, H); 2.10-1.95 (m, 6H); LCMS: 490.36 (M+H).

* cis/trans signals

v. Hydroxylamine solution (1.90 ml of a 50% aqueous solution) was added to a solution of E/Z-1-{4-(2-chloro-4-trifluoromethylphenyloxy)piperidin-1-ylsulphonyl}-5-(pyrimidin-2-yl)pent-1-ene (0.64 g, 1.3 mmol) in THF (9.5 ml) at RT and the mixture stirred overnight. The solvent was reduced in vacuo and the residue partitioned between EtOAc and water. The aqueous layer was extracted with EtOAc and the organics dried (MgSO₄) before being concentrated to a yellow oil to give (R/S)-1-[({4-[2-chloro-4-(trifluoromethyl)phenoxy]piperidin-1-yl}sulphonyl)methyl]-4-pyrimidin-2-ylbutyl(hydroxylamine) (0.67 g, 1.28 mmol). NMR: (CDCl₃, 300 MHz): 8.64 (d, 2H); 7.65 (d, 1Hl); 7.48 (d, 1H); 7.16 (t, 1H); 7.00 (d, 1H); 4.75 (m,1H); 3.60-3.32 (m, 6H, 3.17 (m, 1H); 3.03 (m, 1H); 2.85 (d, 1H); 2.15-1.50 (m, 7H).

§ 4-(2-pyrimidinyl)-butanal has been reported in the literature and has CAS registry number 260441-10-9 (CA Index Name: 2-pyrimidinebutanal).

### Example 3

### (R/S)-1-({[4-(2-bromo-4-fluorophenoxy)piperidin-1-yl]sulphonyl}methyl)-4-pyrimidin-2-ylbutyl(hydroxy)formamide

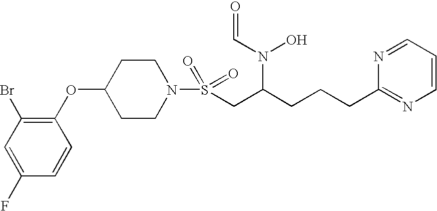

The procedure described in Example 2 was followed using 2-bromo-4-fluorophenol (4.78 g, 25 mmol) in place of 2-chloro-4-trifluoromethylphenol to give (R/S)-1-({[4-(2-bromo-4-fluorophenoxy)piperidin-1-yl]sulphonyl}methyl)-4-pyrimidin-2-ylbutyl(hydroxy)formamide (195 mgs, 0.36 mmol). NMR: 9.7 & 9.35 (d, 1H), 8.5 (d, 2H), 8.14 & 7.7 (d, 1H), 7.35 (m, 1H), 7.1 (t, 1H), 7.0 (m, 2H), 4.4 & 3.9 (br d, 1H), 2.9 (brm, 6H), 2.6 (t, 2M, 1.7 (m, 2H), 1.5 (m, 6H); MS: 545/547.

### Examples 4-21

The procedure described in Example 1 was followed except that the 2-(5-isoxazolyl)-phenol starting material used was replaced by the phenol described.

| Example | Structure and name | Starting phenol | MH+ |
|---|---|---|---|
| 4 | 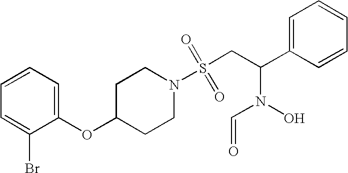 | 2-bromophenol | 483 |
| | (R/S)-2-{[4-(2-bromophenoxy)piperidin-1- | | |
| | yl]sulphonyl}-1-phenylethyl(hydroxy) | | |
| | formamide | | |
| 5 | 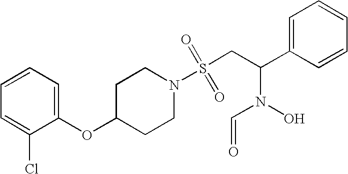 | 2-chlorophenol | 439 |
| | (R/S)-2-{[4-(2-chlorophenoxy)piperidin-1- | | |
| | yl]sulphonyl}-1-phenylethyl(hydroxy) | | |
| | formamide | | |
| 6 | 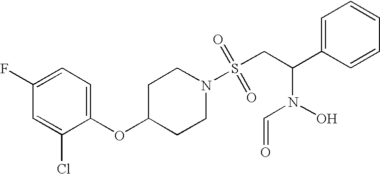 | 2-chloro-4- fluorophenyl | 457 |
| | (R/S)-2-{[4-(2-chloro-4- | | |
| | fluorophenoxy)piperidin-1-yl]sulphonyl}-1- | | |
| | phenylethyl(hydroxy)formamide | | |
| 7 | 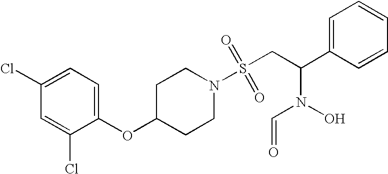 | 2,4-dichlorophenol | 473 |
| | (R/S)-2-{[4-(2,4-dichlorophenoxy)piperidin- | | |
| | 1-yl]sulphonyl}-1-phenylethyl(hydroxy) | | |
| | formamide | | |
| 8 | 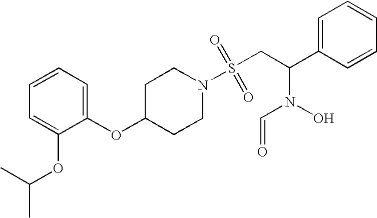 | 2-isopropoxy phenol | 463 |
| | (R/S)-hydroxy(2-{[4-(2- | | |
| | isopropoxyphenoxy)piperidin-1- | | |
| | yl]sulphonyl}-1-phenylethyl)formamide | | |
| 9 | 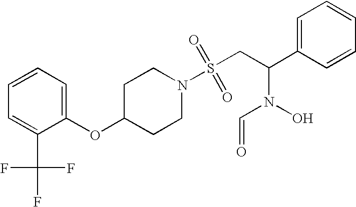 | 2-trifluoromethyl phenol | 473 |
| | (R/S)-hydroxy(2-{[4-(2- | | |
| | trifluoromethylphenoxy)piperidin-1- | | |
| | yl]sulphonyl]-1-phenylethyl)formamide | | |
| 10 | 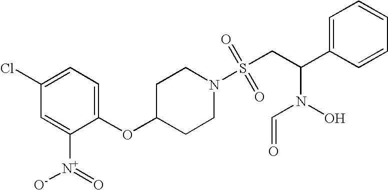 | 4-chloro-2- nitrophenol | 484 |
| | (R/S)-2-{[4-(4-chloro-2- | | |
| | nitrophenoxy)piperidin-1-yl}sulphonyl}-1- | | |
| | phenylethyl(hydroxy)formamide | | |
| 11 | 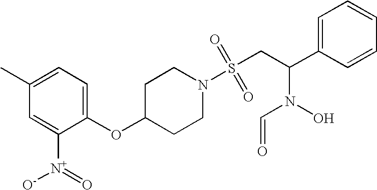 | 4-methyl-2- nitrophenol | 464 |
| | (R/S)-hydroxy(2-{[4-(4-methyl-2- | | |
| | nitrophenoxy)piperidin-1-yl]sulphonyl}-1- | | |
| | phenylethyl)formamide | | |
| 12 | 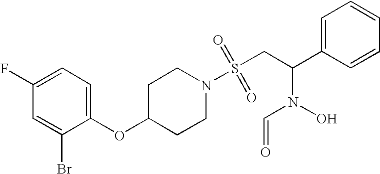 | 4-fluoro-2- bromophenol | 501 |
| | (R/S)-2-{[4-(2-bromo-4- | | |
| | fluorophenoxy)piperidin-1-yl]sulphonyl}-1- | | |
| | phenylethyl(hydroxy)formamide | | |
| 13 | 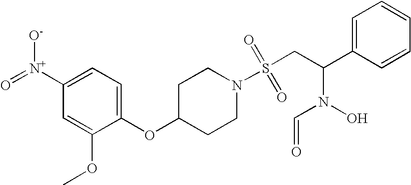 | 2-methoxy-4- nitrophenol | 480 |
| | (R/S)-hydroxy(2-{[4-(2-methoxy-4- | | |
| | nitrophenoxy)piperidin-1-yl]sulphonyl}-1- | | |
| | phenylethyl)formamide | | |
| 14 | 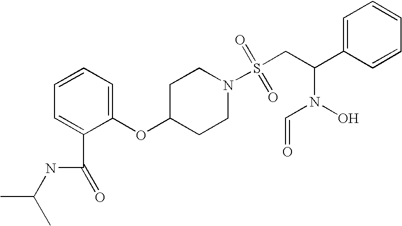 | 2-(isopropylamino carbonyl)-phenol | 490 |
| | (R/S)-hydroxy[2-({4-[2- | | |
| | isopropylaminocarbonyl)phenoxy] | | |
| | piperidin-1-yl}sulphonyl)-1- | | |
| | phenylethyl]formamide | | |
| 15 | 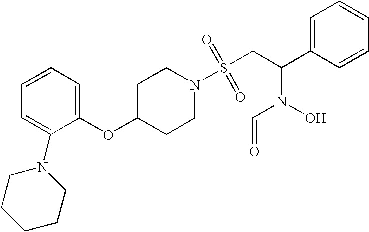 | (2-piperidin-1- yl)phenol | 488 |
| | (R/S)-hydroxy(1-phenyl-2- | | |
| | {[4-(2-piperidin-1-ylphenoxy)piperidin-1- | | |
| | yl]sulphonyl}ethyl)formamide | | |
| 16 | 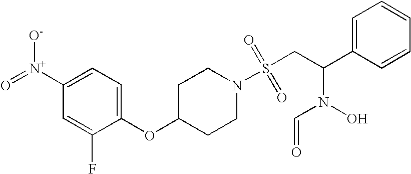 | 2-fluoro-4- nitrophenol | 468 |
| | (R/S)-2-{[4-(2-fluoro-4- | | |
| | nitrophenoxy)piperidin-1-yl]sulphonyl}-1- | | |
| | phenylethyl(hydroxy) | | |
| | formamide | | |
| 17 | 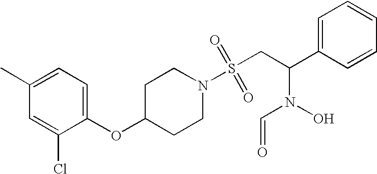 | 2-chloro-4- methylphenol | 453 |
| | (R/S)-2-{[4-(2-chloro-4- | | |
| | methylphenoxy)piperidin-1-yl]sulphonyl}-1- | | |
| | phenylethyl(hydroxy) | | |
| | formamide | | |
| 18 | 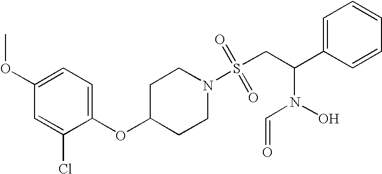 | 2-chloro-4- methoxyphenol | 469 |
| | (R/S)-2-{[4-(2-chloro-4- | | |
| | methoxyphenoxy)piperidin-1-yl]sulphonyl}- | | |
| | 1-phenylethyl(hydroxy) | | |
| | formamide | | |
| 19 | 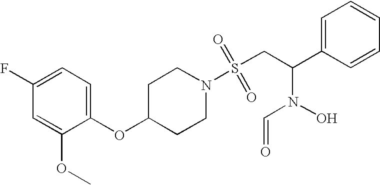 | 4-fluoro-2- methoxyphenol | 453 |
| | (R/S)-2-{[4-(4-fluoro-2- | | |
| | methoxyphenoxy)piperidin-1-yl]sulphonyl}- | | |
| | 1-phenylethyl(hydroxy) | | |
| | formamide | | |
| 20 | 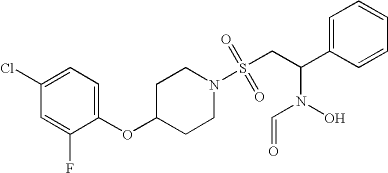 | 4-chloro-2- fluorophenol | 457 |
| | (R/S)-2-{[4-(4-chloro-2- | | |
| | fluorophenoxy)piperidin-1-yl]sulphonyl}-1- | | |
| | phenylethyl(hydroxy) | | |
| | formamide | | |
| 21 | 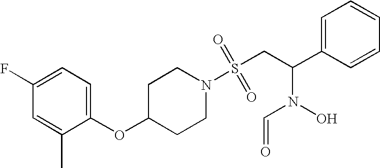 | 4-fluoro-2- methylphenol | 437 |
| | (R/S)-2-{[4-(4-fluoro-2- | | |
| | methylphenoxy)piperidin-1-yl]sulphonyl}-1- | | |
| | phenylethyl(hydroxy)formamide | | |

### Example 22

### (R/S)-2-({4-[(3-chloropyridin-2-yl)oxy]piperidin-1-yl}sulphonyl)-1-pyridin-3-ylethyl(hydroxy)formamide

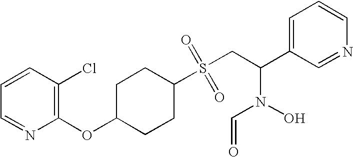

(R/S)-2-({4-[(3-chloropyridin-2-yl)oxy]piperidin-1-yl}sulphonyl)-1-pyridin-3-ylethyl hydroxylamine (0.75 mmol) (prepared below and used directly) was dissolved in DCM (1 ml) and a preformed-mixture of acetic anhydride (1 ml) and formic acid (2 ml) was added before sting at RT overnight. MeOH (5 m]) was then added and, after stirring for 30 minutes, the mixture was evaporated. The residue was re-dissolved in MeOH (2 ml) and allowed to stand at RT overnight. After evaporation the mixture was purified by BondElut chromatography (10 g Silica), eluting with a gradient from DCM to 5% MeOH in DCM to give (R/S)-2-({4[(3-chloropyridin-2-yl)oxy]piperidin-1-yl}sulphonyl)-1-pyridin-3-ylethyl(hydroxy)formamide (47 mgs, 0.11 mmol). MS: 441.

The starting (R/S)-2-({4-[(3-chloropyridin-2-yl)oxy]piperidin-1-yl}sulphonyl)-1-pyridin-3-ylethyl hydroxylamine was prepared as follows:

i) A solution of 4-hydroxypiperidine (8 g; 0.08 mol) in DCM (80 ml) was cooled in an ice bath before pyridine (7.4 ml; 0.09 mol) and TBDMS triflate (20 ml; 0.088 mol) were added. The resulting mixture was stirred for 2½ hours. Iced water was added and the organic layer separated, washed with brine, dried and evaporated to give 4-(tert-butyl dimethylsilyl)oxypiperidine as a pale yellow residue (24 g).

ii) Methanesulphonyl chloride (1.0 ml; 0.012 mol) was added to a solution of the 4-tert-butyl-dimethylsilyloxy-piperidine (2.7 g; 0.012 mol) and DIPEA (4.4 ml; 0.025 mol) in DCM (20 ml) and the whole stirred at RT overnight. Water (20 ml) was added and the organic layer separated and washed with 2M hydrochloric acid, saturated sodium bicarbonate and brine and evaporated to give 1-methanesulphonyl-4-(tert-butyldimethylsilyloxy)piperidine as a black, oily residue.

iii) A solution of 1-methanesulphonyl-4-(tert-butyldimethylsilyloxy)piperidine (2.0 g; 6.8 mmol) in THF (50 ml) was covered with argon and cooled in an ice/acetone bath before a solution of LHMDS in THF (15.0 ml; 1M; 15.0 mmol) was added dropwise. After stirring for 30 minutes, diethyl chlorophosphate (1.0 ml; 6.8 mmol) was added and stirring continued for a further 50 minutes. Nicotinaldehyde (0.64 ml; 6.8 mmol) was then added and the solution allowed to warm to ambient temperature and stirred overnight. A saturated solution of ammonium chloride was then added and the mixture extracted with ethyl acetate. The dried organic extracts were concentrated in vacuo. Purification was by chromatography on silica, eluting with a increasing gradient from hexane to 50% ethyl acetate in hexane to give E-[4-(tert-butyldimethylsilyloxy)piperidin-1-ylsulphonyl]-2-(3-pyridyl)ethene (1.93 g, 5.05 mmol).

iv) E-[4-(tert-butyldimethylsilyloxy)piperidin-1-ylsulphonyl]-2-(3-pyridyl)ethene (1.93 g; 5.05 mmol) was added to a pre-mixture of acetyl chloride (2 ml) in methanol (20 ml) and stirred at room temperature for 2 hours. Concentration in vacuo gave a solid which was partitioned between saturated sodium bicarbonate and ethyl acetate. The organic extracts were dried and evaporated. Purification was by chromatography on silica (20 g) eluting with a gradient from DCM to 20% methanol in DCM. Evaporation of fractions containing product gave E-[4-(hydroxy)piperidin-1-ylsulphonyl]-2-(3-pyridyl)ethene as a white solid (0.4 g; 30%). NMR (400 MHz): 1.4 (2H, m, CH₂); 1.8 (2H, m, CH₂); 2.9 (2H, m, CH₂); 3.2 (2H, m, CH₂); 3.6 (1H, m, CH); 4.8 (1H, d, OH); 7.5 (3H, m, CH); 8.2 (1H, m, CH); 8.6 (1H, m, CH); 8.9 (1H, d, CH).

v) A solution of E-[4-(hydroxy)piperidin-1-ylsulphonyl]-2-(3-pyridyl)ethene in DMF (0.2 g; 0.75 mmol in 3 ml) was added to 2,3-dichloropyridine (1.5 mmol). A covering of argon gas was introduced to the tube before solid sodium hydride (0.1 g incl. oil) was carefully added, in three portions the stirred reaction. Stirring was continued overnight. Water (5 ml) was added (dropwise initially) and the resultant mixture extracted with EtOAc (5 ml). The organic layer was separated and the aqueous layer washed again with EtOAc (3 ml). The combined organics were evaporated, redissolved in DCM (5 ml) and applied to a 10 g Silica BondElut column and eluted with a gradient from DCM to 2.5% MeOH in DCM. Fractions containing pure product were evaporated. This material was dissolved in THF (1 ml) and 50% aqueous hydroxylamine (1 ml) added and the mixture stirred vigorously at RT over night. After partition between water and ethyl acetate, the organic layer was evaporated to dryness to give (R/S)-2-({4-[(3-chloropyridin-2-yl)oxy]piperidin-1-yl}sulphonyl)-1-pyridin-3-ylethyl hydroxylamine.

### Examples 23-29

The procedure described in Example 22 was followed except that the 2,3-dichloropyridine starting material used was replaced by the halo heterocycle described.

| Example | Structure and name | Starting phenol | MH+ |
|---|---|---|---|
| 23 | 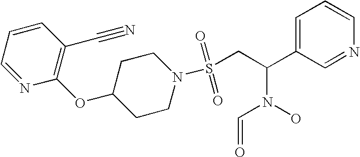 | 2-chloro-3- cyanopyridine | 432 |
| | (R/S)-2-({4-[(3-cyanopyridin-2- | | |
| | yl)oxy]piperidin-1-yl}sulphonyl)-1- | | |
| | pyridin-3-ylethyl(hydroxy)formamide | | |
| 24 | 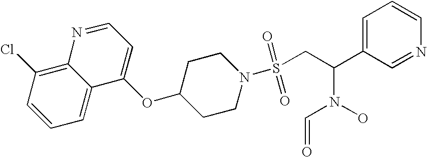 | 4,8-dichloro quinoline | 491 |
| | (R/S)-2-({4-[(8-chloroquinolin-4- | | |
| | yl)oxy]piperidin-1-yl}sulphonyl)-1- | | |
| | pyridin-3-ylethyl(hydroxy)formamide | | |
| 25 | 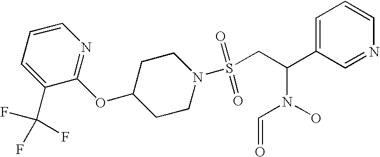 | 2-chloro-3- trifluoromethyl pyridine | 474 |
| | (R/S)-hydroxy{1-pyridin-3-yl-2-[(4-{[3- | | |
| | (trifluoromethyl)pyridin-2-yl]oxy} | | |
| | piperidin-1-yl)sulphonyl]ethyl)formamide | | |
| 26 | 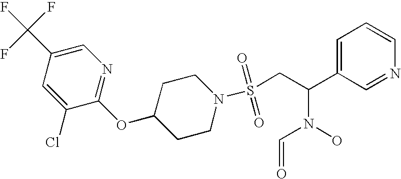 | 2,3-dichloro-5- trifluoromethyl pyridine | 509 |
| | (R/S-2-[(4-{[3-chloro-5- | | |
| | trifluoromethyl)pyridin-2-1]oxy}piperidin- | | |
| | 1-yl)sulphonyl]-1-pyridin-3- | | |
| | ylethyl(hydroxy)formamide | | |
| 27 | 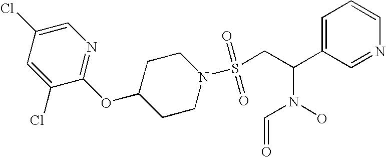 | 2,3,5-trichloro pyridine | 475 |
| | (R/S)-2-[(4-{[3-chloro-5-chloropyridin-2- | | |
| | yl]oxy}piperidin-1-yl)sulphonyl]-1- | | |
| | pyridin-3-ylethyl(hydroxy)formamide | | |
| 28 | 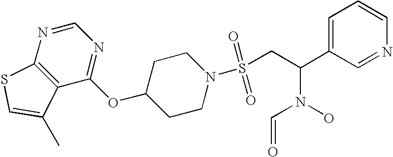 | 4-chloro-5- methylthieno[2,3- d]pyrimidine | 478 |
| | (R/S)-hydroxy[2-({4-[(5-methylthieno[2,3- | | |
| | d]pyrimidin-4-yl)oxy]piperidin-1- | | |
| | yl}sulphonyl)-1-pyridin-3- | | |
| | ylethyl]formamide | | |
| 29 | 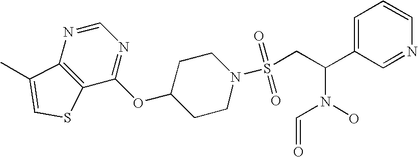 | 4-chloro-7- methylthieno[3,2- d]pyrimidine | 478 |
| | (R/S)-hydroxy[2-({4-[(7-methylthieno[3,2- | | |
| | d]pyrimidin-4-yl)oxy]piperidin-1- | | |
| | yl}sulphonyl)-1-pyridin-3- | | |
| | ylethyl]formamide | | |

### Example 30

### (R/S)-2-({[4-(4-fluoro-2-thien-3-ylphenoxy)piperidin-1-yl]sulphonyl}methyl)-N-hydroxy-4-methylpentanamide

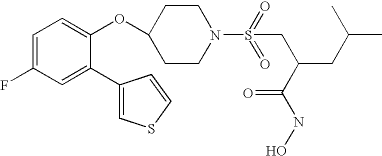

Trimethylaluminium (0.5 ml of a 2M solution in toluene) was added to a suspension of hydroxylamine hydrochloride (65 mg) in toluene at 5° C. under inert atmosphere. The mixture was allowed to warm to RT over 90 minutes before adding a solution of (R/S)-methyl 2-({[4-(4-fluoro-2-thien-3-ylphenoxy)piperidin-1-yl]sulphonyl}methyl)-4-methylpentanoate (80 mg) in dry toluene (1 ml). The mixture was stirred at ambient temperature for 1 hour before partitioning between 2N hydrochloric acid and ethylacetate. The organic phase was dried (Na₂SO₄) and dried under vacuum to yield the product as a yellow gum (48 mg). NMR (CDCl₃): 0.84-0.96 (6H, m), 1.1-1.91 (7H, m), 2.62-2.74 (2H, m), 2.93-3.06 (2H, m), 3.14-3.28 (3H, m), 4.4-4.48 (1H, m), 6.9-6.98 (2H, m), 7.13-7.20 (2H, m), 7.23-7.30 (1H, m), 7.35-7.40 (2H, m), 7.51-7.56 (1H, m)

The starting (R/S)-methyl 2-({[4-(4-fluoro-2-thien-3-ylphenoxy)piperidin-1-yl]sulphonyl}methyl)-4-methylpentanoate was prepared as follows:

i) DIAD (16 ml) was added dropwise to a solution of triphenylphosphine (21.5 g), 2-bromo-4-fluorophenol (15.6 g) and tert-butyl-4-hydroxy-1-piperidinecarboxylate (15 g) in tetrahydrofuran (200 ml) at 20° C. under an inert atmosphere. After stirring at ambient temperature for 4 hours the mixture was partitioned between water and EtOAc. The organic phase was washed with 2N sodium hydroxide solution and water, dried (MgSO₄) and evaporated under vacuum to yield a yellow oil. This was triturated with ether and a white precipitate was isolated by filtration. The filtrates were purified by column chromatography using a gradient of isohexane to 20% EtOAc in isohexane as the eluant to yield tert-butyl 4-(2-bromo-4-fluorophenoxy)piperidine-1-carboxylate as a clear oil (17.2 g). NMR (CDCl₃): 1.49 (9H, s), 1.75-1.95 (4H, m), 3.39-3.49 (2H, m), 3.62-3.74 (2H, m), 4.21-4.29 (1H, m), 6.85-7.01 (2H, m) and 7.26-7.34 (1H, m).

ii) TFA (20 ml) was added to a solution of tert-butyl 4-(2-bromo-4-fluorophenoxy)piperidine-1-carboxylate (19 g) in DCM (200 ml) at ambient temperature. After stirring at RT for 3 hours the mixture was evaporated under vacuum and partitioned between DCM and 2N sodium hydroxide solution. The organic phase was dried (MgSO₄) and evaporated under vacuum to yield 4-(2-bromo-4-fluorophenoxy)piperidine as a clear oil (12.77 g). NMR (CDCl₃) 1.72-1.84 (2H, m), 1.94-2.05 (2H, m), 2.33 (1H, bs), 2.71-2.82 (2H,m), 3.15-3.26 (2H, m), 4.3-4.41 (1H, m), 6.84-7 (2H, m), 7.26-7.33 (1H, m).

iii) Methanesulphonyl chloride (2 ml) was added to a solution of 4-(2-bromo-4-fluorophenoxy)piperidine (6 g) and triethylamine (3.8 ml) in DCM (150 ml) at RT and an exotherm was seen. After stirring at RT for a further 2 hours the mixture was washed with water, dried (MgSO₄), evaporated under vacuum and purified by column chromatography using 50% EtOAc/isohexane as the eluant to yield 4-(2-bromo-4-fluorophenoxy)-1-(methylsulphonyl)piperidine as a white solid (6.5 g). NMR (CDCl₃): 1.92-2.1 (4H, m), 3.02 (3H, s), 3.27-3.39 (2H, m), 2.02-2.11 (2H, m), 4.51-4.6 (1H, m), 6.84-6.9 (1H, m), 6.95-7.03 (1H, m), 7.29-7.33 (1H, m); MS: 352.5/354.5.

iv) LHMDS (13.5 ml) was added over 10 minutes to a solution of 4-(2-bromo-4-fluorophenoxy)-1-(methylsulphonyl)piperidine (4.5 g) in dry THF (45 ml) at −20° C. under an inert atmosphere (solution A). At the same time LHMDS (13.5 ml) was added to a solution of DL-alpha-bromocaprioic acid (2.64 g) in dry THF (35 ml) at −20° C. under inert atmosphere. After sting at −20° C. for a further 10 minutes this was added to solution A and the mixture was allowed to warm to RT over 2½ hours. Aqueous ammoniun chloride solution was added followed by 6N aqueous hydrogen chloride solution until the mixture was just acidic. The reaction mixture was partitioned between water and EtOAc. The organic phase was dried (Na₂SO₄), evaporated under vacuum and purified using a gradient of DCM to 3% MeOH in DCM to yield (R/S)-2-({[4-(2-bromo-4-fluorophenoxy)piperidin-1-yl]sulphonyl}methyl)-4-methylpentanoic acid as a clear oil. NMR (CDCl₃): 0.9-1.1 (6H, m), 1.4-1.51 (1H, m), 1.6-1.73 (2H, m), 1.9-2.0 (4H, m), 2.91-3.09 (2H, m), 3.31-3.55 (5H, m), 4.49-4.57 (1H, m), 6.85-6.91 (1H, m), 6.94-7.02 (1H, m), 7.28-7.32 (1H, m); MS (M−H⁻): 466/464

v) DMF (1 drop) was added to a mixture of (R/S)-2-({[4-(2-bromo-4-fluorophenoxy)piperidin-1-yl]sulphonyl}methyl)-4-methylpentanoic acid (320 mg) and oxalyl chloride (10 ml) in DCM (10 ml). The mixture was sired at RT for 1 hour before evaporating under vacuum to yield a white solid. This was dissolved in MeOH (20 ml, dry) and stirred at RT for 18 hours. The mixture was dried under vacuum and purified by column chromatography using a gradient of DCM to 10% MeOH in DCM as the eluant to yield (R/S)-methyl 2-({[4-(2-bromo-4-fluorophenoxy)piperidin-1-yl]sulphonyl}methyl)-4-methylpentanoate as a clear gum (285 mg). NMR (CDCl₃): 1.9-1.98 (6H, m), 1.34-1.44 (1H, m), 1.52-1.66 (2H,m), 1.92-2.01 (4H, m), 2.87-3.07 (2H, m), 3.39-3.53 (5H, m), 3.74 (3H, s), 4.49-4.55 (1H, m), 6.82-6.89 (1H, m), 6.94-7.01 (1H, m), 7.27-7.32 (1H, m).

vi) A mixture of (R/S)-methyl 2-({[4-(2-bromo-4-fluorophenoxy)piperidin-1-yl]sulphonyl}methyl)-4-methylpentanoate (285 mg), 3-thienylboronic acid (230 mg), water (1 ml) and sodium hydrogen carbonate (150 mg), in dimethoxyethane (10 ml) was degassed with an argon purge before adding Pd(PPh₃)₄ (catalytic). The mixture was heated to 85° C. under argon for 18 hours before partitioning between 2N aqueous HCl and EtOAc. The organic phase was dried (MgSO₄), evaporated under vacuum and purified by column chromatography using a gradient of isohexane to 30% EtOAc in hexane as the eluant to yield (R/S)-methyl 2-({[4-(4-fluoro-2-thien-3-ylphenoxy)piperidin-1-yl]sulphonyl}methyl)-4-methylpentanoate as a yellow gum (196 mg). NMR (CDCl₃): 0.9-1.01 (6H, m), 1.34-1.45 (1H, m), 1.5-1.66 (2H, m), 1.82-1.95 (4H, m), 2.69-2.79 (1H, m), 2.93-3.15 (3H, m), 2.99-3.13 (3H, m), 3.7 (3H, s), 4.4-4.49 (1H, m), 6.9-7 (2H, m), 7.12-7.30 (1H, m), 7.33-7.42 (2H, m), 7.5-7.57 (1H, m); MS: 484.

### Example 31

### (R/S)-2-({[4-(4-fluoro-2-pyridin-3-ylphenoxy)piperidin-1-yl]sulphonyl}methyl)-N-hydroxy-4-methylpentanamide

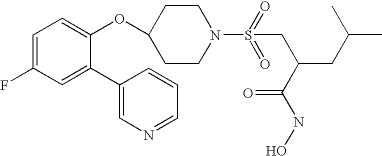

The title compound was made from methyl (R/S)-2-({[4-(4-fluoro-2-pyridin-3-ylphenoxy)piperidin-1-yl]sulphonyl}methyl)-4-methylpentanoate (311 mg) (described below) by the same method as described in Example 30 to yield (R/S)-2-({[4-(4-fluoro-2-pyridin-3-ylphenoxy)piperidin-1-yl]sulphonyl}methyl)-N-hydroxy-4-methylpentanamide as a gum (70 mg). NMR (CDCl₃): 0.94 (6H, d), 1.43-1.94 (7H, m), 2.55-2.63 (1H,m), 2.82-3.03 (3H, m), 3.22-3.42 (1H, m), 3.51-3.66 (2H, m), 4.54-4.6 (1H, m), 6.89-6.91 (1H, m), 7.02-7.12 (2H, m), 7.43-7.5 (1H, m), 7.79 (1H, d), 8.56 (1H, d), 8.94 (1H,s); MS: 480.

The starting (R/S)-2-({[4-(4-fluoro-2-pyridin-3-ylphenoxy)piperidin-1-yl]sulphonyl}methyl)-4-methylpentanoate was prepared as follows:

i) (R/S)-2-({[4-(4-fluoro-2-pyridin-3-ylphenoxy)piperidin-1-yl]sulphonyl}methyl)-4-methylpentanoate was made from (R/S)-methyl 2-({[4-(2-bromo-4-fluorophenoxy)piperidin-1-yl]sulphonyl}methyl)-4-methylpentanoate (750 mg) (described within part v of Example 30) by the same method as for (R/S)-methyl 2-({[4-(4-fluoro-2-thien-3-ylphenoxy)piperidin-1-yl]sulphonyl}methyl)-4-methylpentanoate except 3-thiophene boronic acid was replaced by 3-pyridine boronic acid to yield 480 mgs of product. NMR (CDCl₃): 0.89-0.96 (6H, m), 1.34-2.02 (1H, m), 1.48-1.63 (2H, m), 1.73-2.33 (4H, m), 2.67-2.75 (1H, m), 2.91-3.03 (3H, m), 3.15-3.28 (3H, m), 3.67 (3H, s), 4.35-4.22 (1H, m), 6.93-7.09 (3H, m), 7.33-7.40 (1H, m), 7.80-7.86 (1H, m), 8.59 (1H, dd), 8.78-8.79 (1H, m). MS: 479.

### Example 33

### (R/S)-2-({[4-(2-bromo-4-fluorophenoxy)piperidin-1-yl]sulphonyl}methyl)-N-hydroxy-4-methylpentanamide

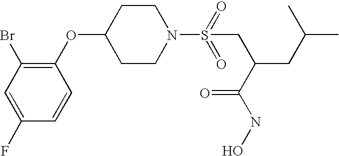

Oxalyl chloride (5 ml) and DM (one drop) were added to a solution of (R/S)-2-({[4-(2-bromo-4-fluorophenoxy)piperidin-1-yl]sulphonyl}methyl)-4-methylpentanoic acid (140 mg) (described within Example 30) in DCM (3 ml). The mixture was stirred at RT for 1½ hours before removing the solvent by evaporation under reduced pressure, redissolving in DCM (5 ml) and adding to a mixture of 50% hydroxylamine in water (0.5 ml) and THF (3 ml). After stirring at RT overnight the mixture was partitioned between ammonium chloride and EtOAc. The organic phase was dried, evaporated under reduced pressure and purified by column chromatography using a gradient of DCM to 3% MeOH in DCM as the eluant to yield (R/S)-2-({[4-(2-bromo-4-fluorophenoxy)piperidin-1-yl]sulphonyl}methyl)-N-hydroxy-4-methylpentanamide as a clear gum (28 mg). NMR (CDCl₃): 0.89-1.01 (6H, m), 1.23-2.07 (7H, m), 2.70-2.96 (2H, m), 3.34-3.51 (5H, m), 4.48-4.57 (1H, m), 6.83-7.03 (2H, m), 7.25-7.36 2H, m); MS (M−H⁻) 497/481.

### Example 34

### (R/S)-3-{[4-(2-bromo-4-fluorophenoxy)piperidin-1-yl]sulphonyl}-2-cyclopentyl-N-hydroxypropanamide

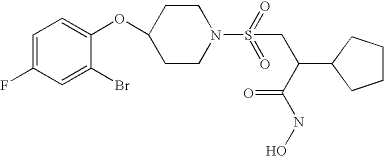

The title compound was made from (R/S)-3-{[4-(2-bromo-4-fluorophenoxy)piperidin-1-yl]sulphonyl}-2-cyclopentylpropanoic acid (described below) using the method described in Example 33 to yield (R/S)-3-{[4-(2-bromo-4-fluorophenoxy)piperidin-1-yl]sulphonyl}-2-cyclopentyl-N-hydroxypropanamide as a white foam (107 mg). NMR: 1.08-1.99 (13H, m), 2.31 (1H, t), 2.98 (1H, d), 3.09-3.52 (5H, m), 4.55-4.64 (1H, m), 7.14-7.25 (2H, m), 7.50-7.57 (1H, m) and 10.525 (1H, bs); MS (M−H⁻): 493.

The starting (R/S)-3-{[4-(2-bromo-4-fluorophenoxy)piperidin-1-yl]sulphonyl}-2-cyclopentylpropanoate was prepared as described below:

i) Sodium metal (2.88 g) was added in small portions to absolute ethanol (220 ml) under argon with stirring at RT. On achievement of complete solution, a mixture of diethyl malonate (20 g) and cyclopentyl bromide (18.64 g) was added and the mixture stirred under reflux for 2 hours, allowed to cool and excess solvent removed in vacuo. The residue was partitioned between water (150 ml) and Et₂O (3×200 ml) and combined organics were dried (sodium sulphate), concentrated in vacuo and purified on a 100 g silica bond elute using a 5-35% EtOAc/isohexane gradient over 50 minutes as eluent to give diethyl cyclopentylmalonate as a colourless oil (18.34 g); NMR: δ 1.1 (m, 9H), 1.5 (m, 4H), 1.7 (m, 2H), 2.3 (m, 1H), 4.1 (q, 4H).

ii) 3M aqueous sodium hydroxide (200 ml) was added to a stirred solution of diethyl cyclopentylmalonate (18.33 g) in THF (300 ml) and MeOH (300 ml). Stirring was continued overnight and organic solvents removed in vacuo. The resulting aqueous solution was saturated with salt, acidified with concentrated hydrochloric acid and partitioned three times with ethyl acetate. The combined organic extracts were dried (MgSO₄), concentrated in vacuo and azeotroped once with toluene to give cyclopentylmalonic acid as an off-white solid (12.7 g); NMR: δ 1.2 (m, 2H), 1.5 (m, 4H), 1.7 (m, 2H), 2.25 (m, 1H), 3.0 (d, 1H), 12.5 (s, 2H); MS: 171.18 (ES−).

iii) Morpholine (7.08 ml) was added to a stirred solution of cyclopentylmalonic acid (12.69 g) in water (55 ml) and acetic acid (9 ml) at RT. After 20 minutes 37% aqueous formaldehyde (3.33 g) was added and stirring was continued overnight. The reaction was then heated to 80° C. and maintained for 2 hours, allowed to cool to RT and made basic with solid sodium hydrogen carbonate. This solution was washed with DCM (100 ml) and then acidified using 2M hydrochloric acid followed by concentrated hydrochloric acid and partitioned with DCM (3×150 ml). Combined organic extracts were washed with water (100 ml) and brine (100 ml), dried (MgSO₄) and concentrated in vacuo to give 2-cyclopentylprop-2-enoic acid as a white solid (2.8 g); NMR: δ 1.3(m, 2H), 1.6(m, 4H), 1.8(m, 2H), 2.85(m, 1H), 5.5(s, 1H), 6.0(s, 1H) and 12.3(s, 1H); MS 139.11 (ES−).

iv) Hydrogen bromide (30 wt. % solution in acetic acid, 22 ml) was added to 2-cyclopentylpropenoic acid (2.8 g). The mixture was stirred at RT overnight and then poured cautiously into water (130 ml) and partitioned with EtOAc (3×75 ml). The combined organic extracts were treated with water (50 ml) and brine (50 ml), dried (MgSO₄), concentrated in vacuo and azeotroped twice with toluene. The crude product was purified on a 50 g silica bond elute using a 25-50% EtOAc/isohexane gradient over 45 minutes as eluent to give (R/S)-3-bromo-2-cyclopentylpropionic acid as a pale yellow solid (2.51 g); NMR: δ 1.2(m, 2H), 1.6(m, 6H), 1.9(m, 1H), 2.5(m, 1H+DMSOd6), 3.6(m, 2H); MS: 223.23(ES+), 221.15 (ES−).

v) (R/S)-3-Bromo-2-cyclopentylpropionic acid (2.5 g) was mixed with DCM (35 ml), isobutylene (18 ml) and concentrated sulphuric acid (2 drops) and the reaction was carried out at 25° C. for 48 hours at a pressure of 1 bar (high pressure facility). The solution was treated with saturated aqueous sodium hydrogen carbonate (50 ml), dried and concentrated in vacuo to give (R/S)-tert-butyl-3-bromo-2-cyclopentylpropionate as a light green oil (1.2 g); NMR: δ 1.3 (m, 3H), 1.4 (s, 9H), 1.6(m, 5H), 1.9 (m, 1H), 2.5(m, 1H), 3.6(m, 2H); MS 278 (ES+).

vi) Potassium thioacetate (1.23 g) was added to a stirred solution of (R/S)-tert-butyl-3-bromo-2-cyclopentylpropionate (1.19 g) in DMF (25 ml) under argon at RT. The solution was heated to 100° C. and maintained for 3 hours, then allowed to cool, poured into water (100 ml) and partitioned with EtOAc (3×150 ml). Combined organic extracts were treated with saturated aqueous sodium hydrogen carbonate (50 ml), water (50 ml) and brine (50 ml), dried (sodium sulphate), concentrated in vacuo and purified on a 20 g silica bond elute using a 0-10% ethyl acetate/isohexane gradient over 45 minutes as eluent to give (R/S)-tert-butyl-3-acetylthio-2-cyclopentylpropionate as a light brown oil (870 mg); NMR: δ 1.2(m, 3H), 1.4(s, 9H), 1.5(m, 4H), 1.8(m, 1H), 1.9(m, 1H), 2.2(m, 1H), 2.3(s, 3H) and 3.0(m, 2H); MS: 273(ES+), 271(ES−).

vii) (R/S)-Tert-butyl-3-acetylthio-2-cyclopentylpropionate (860 mg) was suspended in 5% acetic acid in water (50 ml) and stirred at room temperature. Chlorine gas was bubbled through the suspension for 30 minutes. The chlorine source was then removed and the reaction stirred for a further 30 minutes. The mixture was partitioned with DCM (3×100 ml) and combined organic extracts treated with water (50 ml) and brine (50 ml), dried (magnesium sulphate), concentrated in vacuo and azeotroped once with toluene to give (R/S)-tert-butyl-3-chlorosulphonyl-2-cyclopentylpropionate as a pale yellow oil (930 mg); NMR (CDCl₃): δ 1.4(m, 2H), 1.5(s 9H), 1.7(m, 6H), 2.1(m, 1H), 2.9(m, 1H) and 3.9(m, 2H); MS 296.61(ES+).

viii) (R/S)-Tert-butyl-3-chlorosulphonyl-2-cyclopentylpropionate (486 mg) was added to a mixture of 4-(2-bromo-4-fluorophenoxy)piperidine (430 mg) (described in example 30) and triethylamine (0.22 ml) in DCM at ambient temperature. After stirring at ambient temperature for 18 hours the mixture was washed with water, dried (phase separating cartridge), evaporated under vacuum and purified by column chromatography using a gradient of isohexane to 20% EtOAc/isohexane. (R/S)-Tert-butyl 3-{[4-(2-bromo-4-fluorophenoxy)piperidin-1-yl]sulphonyl}-2-cyclopentylpropanoate was obtained as a white solid (420 mg). NMR (CDCl₃): 1.17-1.85 (17H, m), 1.94-2.05 (5H, m), 2.6-2.7 (1H, m), 2.92-3.01 (1H, m), 3.4-3.58 (5H, m), 4.49-4.56 (1H, m), 6.83-6.91 (1H, m), 6.95-7.02 (1H, m), 7.25-7.34 (1H, m).

ix) TFA (20 ml) was added to a solution of (R/S)-tert-butyl-3-{[4-(2-bromo-4-fluorophenoxy)piperidin-1-yl]sulphonyl}-2-cyclopentylpropanoate (210 mg) in DCM (20 ml). The mixture was stirred at ambient temperature for 3½ hours before removing the solvent by evaporation under reduced vacuum to yield (R/S)-3-{[4-(2-bromo-4-fluorophenoxy)piperidin-1-yl]sulphonyl}-2-cyclopentylpropanoic acid as a gum (175 mg). NMR (CDCl₃): 0.78-2.10 (13H, m), 2.75-2.82 (1H, m), 3.00-3.09 (1H, m), 3.31-3.60 (5H, m), 3.7-3.95 (1H, bs), 4.49-4.56 (1H, m), 6.83-6.90 (1H, m), 6.91-7.01 (1H, m), 7.27-7.33 (1H, m).

### Example 35-38

The method shown in Example 2 was followed except that 2-chloro-4-trifluoromethylphenol was replaced by the appropriate aryl alcohol derivative to give the products shown below.

| Example | Structure and name | Starting phenol | MH+ |
|---|---|---|---|
| 35 | 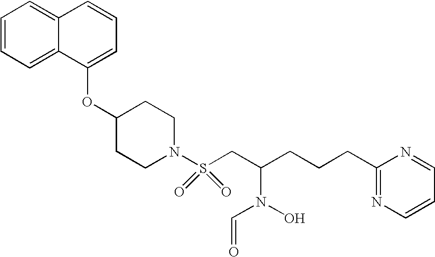 | 1-hydroxy napthalene | 499 |
| | (R/S)-1-[({4-[1-napthyloxy]piperidin-1- | | |
| | yl)sulphonyl)methyl]-4-pyrimidin-2- | | |
| | ylbutyl(hydroxy)formamide | | |
| 36 | 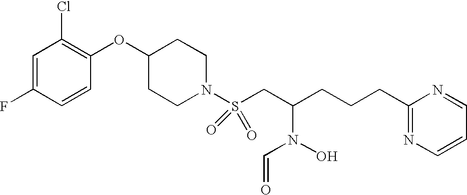 | 2-chloro-4- fluorophenol | 501 |
| | (R/S)-1-[({4-[2-chloro-4- | | |
| | fluorophenoxy]piperidin-1- | | |
| | yl}sulphonyl)methyl]-4-pyrimidin-2- | | |
| | ylbutyl(hydroxy)formamide | | |
| 37 | 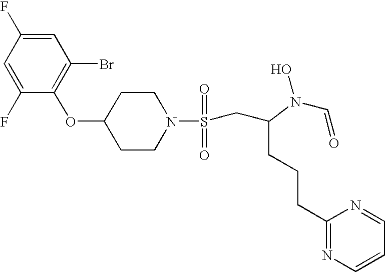 | 2-bromo-4,6- difluorophenol | 563 |
| | (R/S)-1[({4-[2-bromo-4,6- | | |
| | difluorophenoxy]piperidin-1- | | |
| | yl}sulphonyl)methyl]-4-pyrimidin-2- | | |
| | ylbutyl(hydroxy)formamide | | |
| 38 | 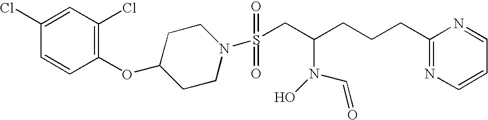 | 2,4-dichloro phenol | 517 |
| | (R/S)-1-[({4-[2,4-dichlorophenoxy]piperidin- | | |
| | 1-yl}sulphonyl)methyl]-4-pyrimidin-2- | | |
| | ylbutyl(hydroxy)formamide | | |

### Example 39

### (R/S)-1-[({4-[2-cyanophenoxy]piperidin-1-yl}sulphonyl)methyl]4-pyrimidin-2-ylbutyl(hydroxy)formamide

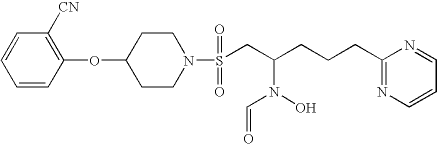

The method described in Example 2 was followed except that 1-tert-butyl-4-(2-chloro-4-trifluoromethylphenyloxy)piperidine carboxylate was replaced by 1-tert-butyl-4-(2-cyanophenyloxy)piperidine carboxylate to give (R/S)-1-[({4-[2cyanophenoxy]piperidin-1-yl}sulphonyl)methyl]-4-pyrimidin-2-ylbutyl(hydroxy)formamide as a cream solid (0.61 g); NMR (CDCl₃): 1.4-1.9 (8H, m), 2.7-2.9 (3H, m), 3.0-3.4 (5H, m), 4.0 (1H, m)*, 4.5 (1H, m), 4.7 (1H, m)*, 6.8 (2H, m), 7.0 (1H, m), 7.3 (2H, m), 7.8 (1H, s)*, 8.2 (1H, s)* and 8.4 (2H, m). MS: 474.

*=rotameric signals.

The starting 1-tert-butyl-4-(2-cyanophenyloxy)piperidine carboxylate was prepared as follows:

i) To a stirred solution of sodium hydride (1.32 g, 33 mmol) in DMF (100 ml) under argon was added 1-tert-butyl-4-hydroxypiperidine carboxylate (5.5 g, 27.5 mmol) followed by 2-fluorobenzenenitrile (3 ml, 27.5 mmol). After 15 hours the mixture was concentrated and ethyl acetate was added. The mixture was washed (water and brine), dried (MgSO4) and concentrated to give 1-tert-butyl-4-(2-cyanophenyloxy)piperidine carboxylate as a crude material (8 g, 26.5 mmol); MS: 203 (MH⁺-Boc).

1. A compound of formula (1): 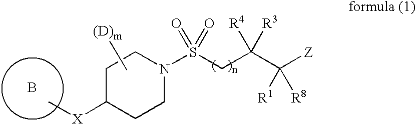
wherein:
Z is selected from —CONR¹⁵OH and —N(OH)CHO;
R¹⁵ is hydrogen or C₁₋₃alkyl;
R¹ is hydrogen or a group selected from C₁₋₆alkyl, C₂₋₆alkenyl, C₂₋₆alkynyl, C₃₋₇cycloalkyl, C₅₋₇cycloalkenyl, aryl and heteroaryl where the group is optionally substituted by one or more substituents independently selected from halo, nitro, cyano, trifluoromethyl, trifluoromethoxy, C₁₋₄alkyl, C₂₋₄alkenyl, C₂₋₄alkynyl, C₃₋₆cycloalkyl (optionally substituted by one or more R¹⁷), aryl (optionally substituted by one or more R¹⁷), heteroaryl (optionally substituted by one or more R¹⁷), heterocyclyl, C₁₋₄alkoxycarbonyl, —OR⁵, —SR², —SOR², —SO₂R², —COR², —CO₂R⁵, —CONR⁵R⁶, —NR¹⁶COR⁵, —SO₂NR⁵R⁶ and —NR¹⁶SO₂R²;
R¹⁶ is hydrogen or C₁₋₃alkyl;
R¹⁷ is selected from halo, C₁₋₆alkyl, C₃₋₆cycloalkyl and C₁₋₆alkoxy;
R² is group selected from C₁₋₆alkyl, C₃₋₆cycloalkyl, C₅₋₇cycloalkenyl, heterocycloalkyl, aryl, heteroaryl, arylC₁₋₄alkyl and heteroarylC₁₋₄alkyl where the group is optionally substituted by one or more halo;
R⁵ is hydrogen or a group selected from C₁₋₆alkyl, C₃₋₆cycloalkyl, C₅₋₇cycloalkenyl, heterocycloalkyl, aryl, heteroaryl, arylC₁₋₄alkyl and heteroarylC₁₋₄alkyl where the group is optionally substituted by one or more halo;
R⁶ is hydrogen, C₁₋₆alkyl or C₃₋₆cycloalkyl;
or R⁵ and R⁶ together with the nitrogen to which they are attached form a heterocyclic 4- to 7-membered ring;
R⁸ is hydrogen or a group selected from C₁₋₆alkyl, C₃₋₇cycloalkyl and C₅₋₇cycloalkenyl where the group is optionally substituted by one or more substituents independently selected from halo, nitro, cyano, trifluoromethyl, trifluoromethoxy and C₁₋₄alkyl;
R³ and R⁴ are both hydrogen;
n is 0 or 1;
m is 0 or 1;
D is hydrogen, C₁₋₄alkyl, C₃₋₆cycloalkyl or fluoro;
X is O, S, SO or SO₂;
B is monocyclic aryl or heteroaryl where each is substituted in an ortho position by, and is optionally further substituted by one or more groups independently selected from nitro, trifluoromethyl, trifluoromethoxy, halo, C₁₋₄alkyl (optionally substituted by R¹³), C₂₋₄alkenyl (optionally substituted by R¹³), C₂₋₄alkynyl (optionally substituted by R¹³), C₃₋₆cycloalkyl (optionally substituted by R¹³), C₃₋₆cycloalkenyl (optionally substituted by R¹³), phenyl (optionally substituted by halo or C₁₋₄alkyl), heteroaryl (optionally substituted by halo or C₁₋₄alkyl), heterocyclyl (optionally substituted by halo or C₁₋₄alkyl), C₁₋₄alkylthio, C₃₋₆cycloalkylthio, —SOR¹³, —SO₂R¹³, —SO₂NHR¹³, —SO₂NR¹³R¹⁴, —NHSO₂R¹³, —NR¹³SO₂R¹⁴, —NHCONHR¹³, —NHCONHR¹³R¹⁴, —OR¹³, cyano, —CONR¹³R¹⁴, —NHCOR¹³, —CO²R¹³ and —CH₂CO₂R¹³;
or B is bicyclic aryl or heteroaryl where each is optionally substituted by one or more groups independently selected from nitro, trifluoromethyl, trifluoromethoxy, halo, C₁₋₄alkyl (optionally substituted by R¹³), C₂₋₄alkenyl (optionally substituted by R¹³), C₂₋₄alkynyl (optionally substituted by R¹³), C₃₋₆cycloalkyl (optionally substituted by R¹³), C₃₋₆cycloalkenyl (optionally substituted by R¹³), C₁₋₄alkylthio, C₃₋₆cycloalkylthio, —SOR¹³, —SO₂R¹³, —SO₂NHR¹³, —SO₂NR¹³R¹⁴, —NHSO₂R¹³, —NR¹³SO₂R¹⁴, —NHCONHR¹³, —NHCONHR¹³R¹⁴, —OR¹³, cyano, —CONR¹³R¹⁴ and —NHCOR¹³;
R¹³ and R¹⁴ are independently hydrogen, C₁₋₆alkyl or C₃₋₆cycloalkyl;
or R¹³ and R¹⁴ together with the nitrogen to which they are attached form a heterocyclic 4 to 7-membered ring.
or a pharmaceutically acceptable salt thereof.

2. A compound according to claim 1 wherein B is phenyl or pyridyl where each is substituted in an ortho position by, and is optionally further substituted by one or more groups independently selected from halo, trifluoromethyl, cyano, C₁₋₄alkoxy, C₁₋₄alkyl, nitro, aryl, heteroaryl, heterocyclyl, N—(C₁₋₄alkyl)carbamoyl and N,N—(C₁₋₄alkyl)₂carbamoyl; or B is naphthyl, quinolinyl, thieno[2,3-d]pyrimidinyl or thieno[3,2-d]pyrimidinyl each being optionally substituted by one or more groups independently selected from halo, trifluoromethyl, cyano, C₁₋₄alkoxy, C₁₋₄alkyl, aryl, heteroaryl, heterocyclyl and nitro.

3. A compound according to claim 1 wherein R¹ is a group selected from C₁₋₆alkyl, C₃₋₆cycloalkyl, aryl, heteroaryl and C₁₋₆alkyl substituted by aryl or heteroaryl wherein any R¹ group is optionally substituted by one or more substituents independently selected from halo, C₁₋₄alkoxy, C₁₋₄alkyl and C₃₋₆cycloalkyl.

4. A compound according to claim 1 wherein X is O.

5. (canceled)

6. A method, the method comprising treating a disease condition mediated by one or more metalloproteinase enzymes by administering to a warm-blooded animal a compound according to claim 1.

7. A method, the method comprising treating a disease condition mediated by TNFα, by administering to a warm-blooded animal a compound according to claim 1.

8. A pharmaceutical composition comprising a compound according to claim 1; and a pharmaceutically-acceptable diluent or carrier.

9. A method of treating autoimmune disease, allergic/atopic diseases, transplant rejection, graft versus host disease, cardiovascular disease, reperfusion injury and malignancy in a warm-blooded animal, such as man, in need of such treatment which comprises administering to said animal an effective amount of a compound according to claim 1.

10. A process for preparing a compound of formula (1) according to claim 1 comprising, when Z is —N(OH)CHO, the step of:
a) converting a hydroxylamine of formula (2) into a compound of formula (1); 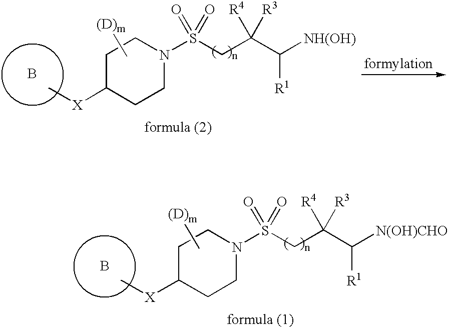
or when Z is —CONR¹⁵OH, the step of:
b) converting an acid of formula (14) into a compound of formula (1); 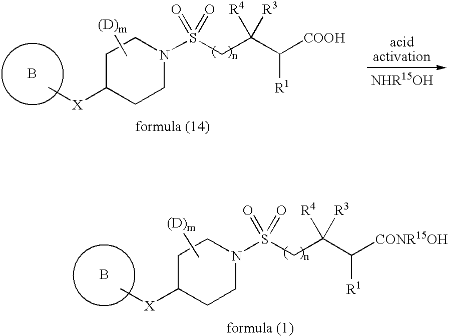
and thereafter if necessary:
i) converting a compound of formula (1) into another compound of formula (1);
ii) removing any protecting groups;
iii) forming a pharmaceutically acceptable salt or in vivo hydrolysable ester.
